(19) United States
(12) Patent Application Publication
(10) Pub. No.: US 2022/0344014 A1
(43) Pub. Date: Oct. 27, 2022

(54) INFORMATION PROCESSING METHOD, STORAGE MEDIUM, AND INFORMATION PROCESSING DEVICE

(71) Applicant: NIPPON TELEGRAPH AND TELEPHONE CORPORATION, Tokyo (JP)

(72) Inventors: Sun Yeong KIM, Tokyo (JP); Atsuhiko MAEDA, Tokyo (JP); Kenichi FUKUDA, Tokyo (JP); Yukio KIKUYA, Tokyo (JP); Kazuaki OBANA, Tokyo (JP)

(73) Assignee: NIPPON TELEGRAPH AND TELEPHONE CORPORATION, Tokyo (JP)

(21) Appl. No.: 17/773,474

(22) PCT Filed: Nov. 1, 2019
(86) PCT No.: PCT/JP2019/043080

§ 371 Date: Apr. 29, 2022

(51) Int. Cl. G16H 10/60; G16H 40/20; G06K 9/62

(52) U.S. Cl. CPC G16H 10/60; G16H 40/20; G06K 9/628; G06K 9/6288

(57) ABSTRACT

Provided is a technique for efficiently predicting the number of occurrences of emergency medical service requests in a target area. At least patient attributes being attributes of patients, date/time information indicating dates and times of occurrences of emergency medical service requests, position information indicating places of the occurrences of the emergency medical service requests, and illness/injury information indicating illnesses and injuries that caused the emergency medical service requests are obtained. The illnesses and injuries are categorized into illness/injury groups, on a basis of a likelihood of occurrences of the illnesses and injuries in the illness/injury information obtained by bringing the patient attributes, the position information, and the date/time information into association with one another. A quantity of occurrences of emergency medical service requests is estimated by bringing an arbitrary date/time into association with frequency of occurrences of each of the illness/injury groups calculated for each prescribed unit area and each prescribed unit time period, by using a first method. The quantity of the occurrences of the emergency medical service requests is output.

### TECHNICAL FIELD

An aspect of the present disclosure relates to an information processing method, a storage medium, and an information processing device for predicting the number of occurrences of emergency medical service requests in emergency medicine.

### BACKGROUND ART

Along with the population aging of recent years, the number of emergency medical staff dispatches triggered by the 119 calls from citizens in Japan is increasing year by year. In addition, working periods of the dispatched emergency medical staff also have a tendency of increasing. In actual paramedic situations, a delay of a few minutes can be fatal. However, because a budget allocated to the fire stations is limited, the number of emergency medical staff is not expected to increase significantly. It is therefore an urgent task to develop a technique for reducing the working periods of the dispatched emergency medical staff.

For example, in the City of Sapporo, an attempt has been made to predict the demand for emergency medical services in the future for each municipal ward, by multiplying a predicted future increase in the population by the numbers of emergency medical transports in different municipal wards (Central Ward, North Ward, East Ward, etc.) and age groups in five-year increments (ages 0-4, 5-9, 10-14, and so on), so as to explore an optimal allocation of ambulances (Non-Patent Literature 1). Further, the Japanese Ministry of Internal Affairs and Communications publicized introducing a system related to a prediction of the demand for emergency medical services made by using big data and to efficient allocations of ambulances based on the prediction (Non-Patent Literature 2).

### CITATION LIST

### Non-Patent Literature

Non-Patent Literature 1: Kyukyutai no Tekisei Haichi Nado ni Kansuru Kenkyukai (Sapporo Shi Shobokyoku), Heisei 26 Nendo Ippan Zaidan Hojin Kyukyu Shinko Zaidan Chosa Kenkyu Josei Jigyo “Kyukyu Juyo Zoka ni Tomonau Kyukyutai no Tekisei Haichi Nado ni Kansuru Kenkyu ni Tsuite” [A Study Group on an Optimal Allocation of Emergency Medical Staff and others (The Fire Department of The City of Sapporo) A 2014 Research Subsidy Program of the General Incorporated Foundation, Foundation for Ambulance Service Development, “A Study on an Optimal Allocation of Emergency Medical Staff and Others in Response to an Increase in the Demand for Emergency Medical Services” (in Japanese)], [online], March 2015, Internet <URL: http://www.fasd.or.jp/tyousa/pdf/h26tekisei.pdf>
Non-Patent Literature 2: The Nikkei Newspaper, Electronic Edition, “Kyukyusha o Koritsu Haichi, Big Data Katsuyo, Juyo Yosoku” [Efficient Allocation of Ambulances, Utilizing Big Data to Predict the Demand (in Japanese)] [online], Oct. 28, 2016, Internet <URL: https://www.nikkei.com/article/DGXLASFS31H0P_Y6A021C1MM00 00/>

### SUMMARY OF THE INVENTION

### Technical Problem

According to the techniques described in the listed non-patent literature, in order to optimally allocate the ambulances, it is necessary to predict the demand for emergency medical services on the level of small regions (e.g., the Japanese ‘chome’ (address blocks) or tertiary meshes). However, as the region is divided into small sections, the frequency of occurrences of emergency medical service requests also decreases. Analyses as probability events are therefore extremely difficult.

In this regard, the number of occurrences of emergency medical service requests is considered to be impacted by various environmental factors. Examples of the environmental factors include the following:
Climate information such as temperature, humidity, atmospheric pressure, and the like (heatstroke, migraine, asthma, arthralgia, etc.)
Weather information (traffic accidents due to rainfalls, people slipping on snow-covered ground, etc.)
epidemic situations of infectious diseases (colds, influenza, etc.)
characteristics unique to certain regions (acute alcoholism in commercial districts, etc.).

It is considered to enhance the precision of an estimation of the demand for emergency medical services, by making clear the various environmental factors and groups of illnesses and injuries impacted by those factors. In emergency medical transport data, however, because the types of illnesses and injuries are categorized in extremely small divisions, generally speaking, many illnesses and injuries do not exhibit sufficiently high frequency of occurrences that withstand analyses. Further, different municipalities use different formats for emergency medical transport data. Also, different municipalities use different categorizations of illnesses and injuries. It is therefore not easy to generalize the information.

Further, the number of emergency medical transports fluctuates from season to season and particularly increases in the summer and the winter when meteorological conditions are harsher. Various illnesses and injuries including cerebral diseases and psychogenic issues seem to have a seasonal increase. In particular, outbursts of sudden increases often occur due to drastic changes in meteorological situations, such as heatstroke in the summer and an epidemic of influenza and people slipping or having bone fractures due to snowfall or frozen surfaces in the winter. Generally speaking, in the actual scenes of emergency medical services, there is a need especially for a capability to predict outbursts of such sudden increases in the emergency medical transports in the summer and winter seasons.

However, those sudden increases in the emergency medical transports caused by the drastic meteorological changes are rare phenomena from a point of view over the entire year. Accordingly, when a prediction model for an entire year is constructed by machine learning, those sudden increases are likely to be easily ignored and tend to be not reflected in the learning.

In view of the circumstances described above, it is an object of the present disclosure to provide a technique for predicting the number of occurrences of emergency medical service requests, with a high level of precision and efficiently.

### Means for Solving the Problem

A first aspect of the present disclosure provides an information processing method implemented by a processor that executes an instruction stored in a storage device. As a result of the processor executing the instruction stored in the storage device, a computer executes a process including: obtaining at least patient attributes being attributes of patients, date/time information indicating dates and times of occurrences of emergency medical service requests, position information indicating places of the occurrences of the emergency medical service requests, and illness/injury information indicating illnesses and injuries that caused the emergency medical service requests; categorizing the illnesses and injuries into illness/injury groups, on a basis of a likelihood of occurrences of the illnesses and injuries in the illness/injury information obtained by bringing the patient attributes, the position information, and the date/time information into association with one another; estimating a quantity of occurrences of emergency medical service requests by bringing an arbitrary date/time into association with frequency of occurrences of each of the illness/injury groups calculated for each prescribed unit area and each prescribed unit time period, by using a first method; and outputting the quantity of the occurrences of the emergency medical service requests.

A second aspect of the present disclosure provides a storage medium storing therein an instruction that is stored in a storage unit and to be executed by a processor. As a result of the processor executing the instruction stored in the storage unit, a computer is caused to execute: obtaining at least patient attributes being attributes of patients, date/time information indicating dates and times of occurrences of emergency medical service requests, position information indicating places of the occurrences of the emergency medical service requests, and illness/injury information indicating illnesses and injuries that caused the emergency medical service requests; categorizing the illnesses and injuries into illness/injury groups, on a basis of a likelihood of occurrences of the illnesses and injuries in the illness/injury information obtained by bringing the patient attributes, the position information, and the date/time information into association with one another; estimating a quantity of occurrences of emergency medical service requests by bringing an arbitrary date/time into association with frequency of occurrences of each of the illness/injury groups calculated for each prescribed unit area and each prescribed unit time period, by using a first method; and outputting the quantity of the occurrences of the emergency medical service requests.

A third aspect of the present disclosure provides an information processing device that performs processing of processing units by employing a processor that executes an instruction stored in a storage unit. As a result of the processor executing the instruction stored in the storage unit, the information processing device includes: a pre-processing unit that obtains at least patient attributes being attributes of patients, date/time information indicating dates and times of occurrences of emergency medical service requests, position information indicating places of the occurrences of the emergency medical service requests, and illness/injury information indicating illnesses and injuries which caused the emergency medical service requests and that categorizes the illnesses and injuries into illness/injury groups, on a basis of a likelihood of occurrences of the illnesses and injuries in the illness/injury information obtained by bringing the patient attributes, the position information, and the date/time information into association with one another; and a prediction unit that estimates a quantity of occurrences of emergency medical service requests by bringing an arbitrary date/time into association with frequency of occurrences of each of the illness/injury groups calculated for each prescribed unit area and each prescribed unit time period, by using a first method and that outputs the quantity of the occurrences of the emergency medical service requests.

### Effects of the Invention

It is possible to provide the technique for predicting the number of occurrences of the emergency medical service requests, with a high level of precision and efficiently.

### BRIEF DESCRIPTION OF DRAWINGS

FIG. 1 is a block diagram showing a first example of a functional configuration of an emergency medical service demand prediction device according to a reference example of the present disclosure.

FIG. 2 is a flowchart showing an example of a categorization model learning procedure performed by the emergency medical service demand prediction device shown in FIG. 1.

FIG. 3 is a flowchart showing an example of a prediction model learning procedure performed by the emergency medical service demand prediction device shown in FIG. 1.

FIG. 4 is a flowchart showing an example of a predicting procedure performed by the emergency medical service demand prediction device shown in FIG. 1.

FIG. 5 is a table showing an example of actual history data of emergency medical transports.

FIG. 6A is a table showing an example of a result of extracting illness/injury groups by using a clustering scheme.

FIG. 6B is a chart showing an example of visualizing the result of extracting the illness/injury groups by using the clustering scheme.

FIG. 6C is a table of a list showing an example in which results of a clustering process are visualized.

FIG. 7 is a table showing an example of prediction model learning-purpose data.

FIG. 8 is a table showing an example of a prediction result obtained by the emergency medical service demand prediction device shown in FIG. 1.

FIG. 9 is a block diagram showing a second example of the functional configuration of the emergency medical service demand prediction device according to a reference example of the present disclosure.

FIG. 10 is a table showing an example of environment data.

FIG. 11 is a table showing an example of environment data on which pre-processing processes have been performed.

FIG. 12 is a block diagram showing a third example of the functional configuration of the emergency medical service demand prediction device according to a reference example of the present disclosure.

FIG. 13 is a table showing an example of region data.

FIG. 14 is a block diagram showing a fourth example of the functional configuration of the emergency medical service demand prediction device according to a reference example of the present disclosure.

FIG. 15 is a table showing an example of regional characteristic learning-purpose data.

FIG. 16 is a table showing an example of regional characteristic learning results.

FIG. 17 is a flowchart showing a second example of a learning procedure performed by the emergency medical service demand prediction device according to a reference example of the present disclosure.

FIG. 18 is a flowchart showing a second example of the predicting procedure performed by the emergency medical service demand prediction device according to a reference example of the present disclosure.

FIG. 19 is a table showing an example of learning-purpose data used in the learning procedure shown in FIG. 17.

FIG. 20 is a schematic chart showing data flows in the learning procedure in FIG. 17 and the predicting procedure in FIG. 18.

FIG. 21A is a drawing showing a first example of visually displaying results of predicting an emergency medical service demand.

FIG. 21B is a drawing showing a second example of visually displaying results of predicting an emergency medical service demand.

FIG. 22 is a drawing showing examples of prediction results obtained when a vector regression model is used as a prediction model.

FIG. 23 is a drawing showing examples of prediction results obtained when a deep learning model is used as a prediction model.

FIG. 24 is a block diagram showing an example of a functional configuration of an emergency medical service demand prediction device according to one embodiment of the present disclosure.

FIG. 25 is a block diagram showing a detailed configuration of a prediction model learning unit according to one embodiment of the present disclosure.

FIG. 26 is a flowchart showing a learning process performed by an emergency medical service demand prediction device 1 according to one embodiment of the present disclosure.

FIG. 27 is a flowchart showing a predicting process performed by the emergency medical service demand prediction device 1 according to one embodiment of the present disclosure.

### DESCRIPTION OF EMBODIMENTS

With reference to the drawings, the following will describe reference examples used as a presupposition of embodiments of the present disclosure, before describing the embodiments of the present disclosure.

### REFERENCE EXAMPLES

### First Reference Example

<A configuration>

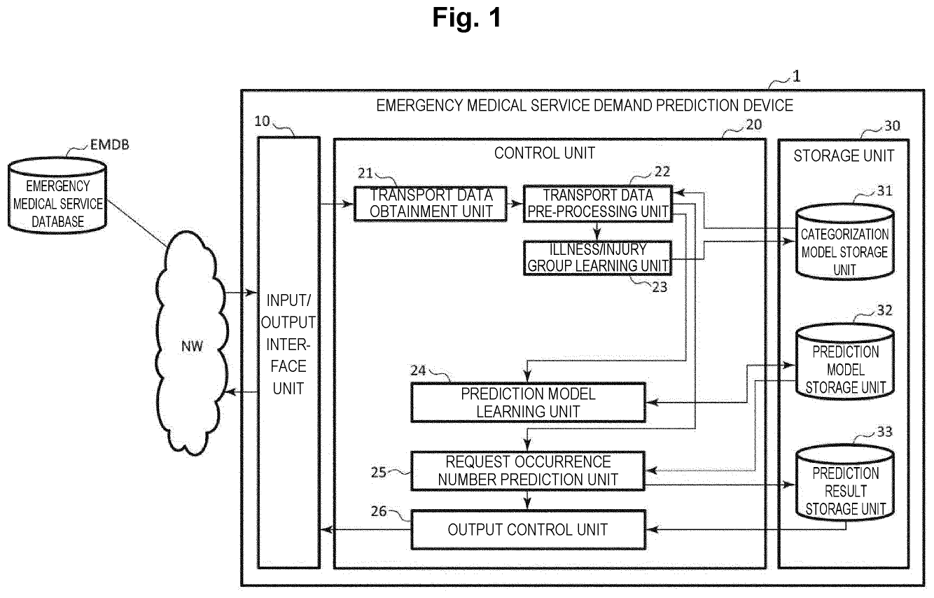

FIG. 1 is a block diagram showing a functional configuration of an emergency medical service demand prediction device 1 according to a reference example of the present disclosure.

The emergency medical service demand prediction device 1 is managed by the fire department headquarter of each municipality, for example, and is configured by using a server computer or a personal computer. The emergency medical service demand prediction device 1 is configured to estimate the number of occurrences of emergency medical services per unit time period per unit area, for a number of hours or days later, by using the number of occurrences of emergency medical service requests per unit area in the past as training data.

The emergency medical service demand prediction device 1 is capable of communicating with various types of servers and databases such as an emergency medical service database EMDB, via a network NW. For example, the emergency medical service database EMDB has accumulated therein data related to an actual history of occurrences of emergency medical service requests, including emergency medical transport information and patient information input by control offices and emergency medical staff.

The network NW is structured with, for example, a relay network and a plurality of access networks for accessing the relay network. Examples of types of the access networks include public networks such as the Internet being commonly used and closed networks that are controlled so that only limited devices can have access thereto. As the relay network, for example, a public network or a closed network using an internet protocol may be used. As the access networks, for example, Local Area Networks (LANs), wireless LANs, mobile phone networks, wired phone networks, Fiber To The Home (FTTH) systems, or Cable Television (CATV) networks, may be used.

The emergency medical service demand prediction device 1 according to a reference example includes an input/output interface unit 10, a control unit 20, and a storage unit 30.

The input/output interface unit 10 includes, for example, at least one wired or wireless communication interface unit and makes it possible to transmit and receive information to and from external devices. Examples of the wired interface include a wired LAN. Examples of the wireless interface include an interface using a low-power wireless data communication standard such as a wireless LAN or Bluetooth (registered trademark).

For example, under control of the control unit 20, the input/output interface unit 10 performs a process of accessing the emergency medical service database EMDB, reading any of the accumulated data, and further forwarding the read data to the control unit 20. Further, the input/output interface unit 10 is also capable of performing a process of outputting instruction information input through an input device (not shown) such as a keyboard, to the control unit 20. Further, the input/output interface unit 10 is capable of performing a process of outputting a learning result or a prediction result output from the control unit 20 to a display device (not shown) such as a liquid crystal display device or transmitting those results to an external device via the network NW.

The storage unit 30 uses, as a storage medium thereof, a non-volatile memory such as a Hard Disk Drive (HDD) or a Solid State Drive (SSD), for example, to and from which it is possible to write and read data when necessary. Further, as storage areas necessary for realizing the present reference example, the storage unit 30 includes, in addition to a program storage unit, a categorization model storage unit 31, a prediction model storage unit 32, and a prediction result storage unit 33.

The categorization model storage unit 31 is used for storing therein a categorization model for re-categorizing illness/injury categories into groups of illnesses and injuries (hereinafter “illness/injury groups”) on the basis of occurrence patterns.

The prediction model storage unit 32 is used for storing therein a prediction model for predicting the number of occurrences of emergency medical service requests in the future on the basis of actual history data from the past.

The prediction result storage unit 33 is used for storing therein a prediction result obtained by using the prediction model that has been trained (hereinafter, “trained prediction model”).

It should be noted, however, that the storage units 31 to 33 are not requisite configurations and may be provided, for example, in an external storage medium such as a USB memory or in a storage device such as a database server placed in a cloud.

The control unit 20 includes (not shown) a hardware processor such as a Central Processing Unit (CPU) or a Micro Processing Unit (MPU) and a memory such as a Dynamic Random Access Memory (DRAM) or a Static Random Access Memory (SRAM), and further includes, as processing functions necessary for carrying out the present reference example, a transport data obtainment unit 21, a transport data pre-processing unit 22, an illness/injury group learning unit 23, a prediction model learning unit 24, a request occurrence number prediction unit 25, and an output control unit 26. All of these processing functions are realized as a result of causing the abovementioned processor to execute a program stored in the storage unit 30. Alternatively, the control unit 20 may be realized in any of other various forms including an integrated circuit such as an Application Specific Integrated Circuit (ASIC) or a Field-Programmable Gate Array (FPGA). The program may be executed by a combination of two or more processors of mutually the same type or mutually-different types (e.g., a plurality of FPGAs or a combination of a CPU and an FPGA). Further, the hardware structure of each of these types of processors is, more specifically, an electric circuit combining together circuit elements such as semiconductor elements. In an example, the program may be provided while being stored in a non-transitory storage medium such as a Compact Disk Read Only Memory (CD-ROM), a Digital Versatile Disk Read Only Memory (DVD-ROM), or a Universal Serial Bus (USB) memory. In yet another example, the program may be downloaded from an external device via a network.

The transport data obtainment unit 21 performs a process of obtaining, from the external emergency medical service database EMDB via the input/output interface unit 10, actual history data of emergency medical transports (hereinafter, “transport data”) from the past that was recorded every time emergency medical staff was dispatched and further forwarding the obtained data to the transport data pre-processing unit 22.

The transport data pre-processing unit 22 performs a process of performing a pre-processing process on the received transport data and subsequently forwarding the resulting data to one selected from among the illness/injury group learning unit 23, the prediction model learning unit 24, and the request occurrence number prediction unit 25. For example, on the basis of the received transport data, the transport data pre-processing unit 22 generates one of illness/injury group learning-purpose data, prediction model learning-purpose data, or request occurrence number prediction-purpose data, by dividing the received data into sections, extracting necessary items, supplementing missing information, and performing a normalization process and further forwards the generated data to the units 23 to 25. Further, when both the illness/injury group learning-purpose data and the prediction model learning-purpose data are referred to, the two types of data will simply be referred to as learning-purpose data.

The illness/injury group learning unit 23 performs a process of receiving the illness/injury group learning-purpose data from the transport data pre-processing unit 22, further learning illness/injury groups each exhibiting similar occurrence patterns, and saving the learning result into the categorization model storage unit 31.

The prediction model learning unit 24 performs a process of receiving the prediction model learning-purpose data from the transport data pre-processing unit 22, further learning the prediction model for predicting the total number of occurrences of emergency medical service requests on the basis of the number of occurrences and date/time information of each illness/injury group, and saving the learning result into the prediction model storage unit 32.

The request occurrence number prediction unit 25 performs a process of predicting the number of occurrences of emergency medical service requests for each unit area and saving the prediction result into the prediction result storage unit 33. The predicting process is performed by receiving the request occurrence number prediction-purpose data from the transport data pre-processing unit 22, further reading the trained prediction model saved in the prediction model storage unit 32, and inputting the request occurrence number prediction-purpose data to the prediction model.

The output control unit 26 performs a process of generating output data on the basis of the prediction result from the request occurrence number prediction unit 25 and further outputting the output data via the input/output interface unit 10. For example, the output control unit 26 is capable of generating the output data for causing a display device (not shown) to display the prediction number for each unit area as a two-dimensional map and outputting the generated output data to the display device. Also, the output control unit 26 is capable of generating the output data on the basis of the data stored in the categorization model storage unit 31, the prediction model storage unit 32, and the prediction result storage unit 33.

<An Operation>

Next, an operation of the emergency medical service demand prediction device 1 configured as described will be explained.

For example, the emergency medical service demand prediction device 1 is capable of starting a learning process or a predicting process, upon receipt of an instruction signal from an operator or the like that is input via an input device (not shown).

(1) An Illness/Injury Group Learning Process

Upon receipt of an instruction signal for an illness/injury group learning process, the emergency medical service demand prediction device 1 performs a process of learning the categorization model of the illness/injury groups, as described below.

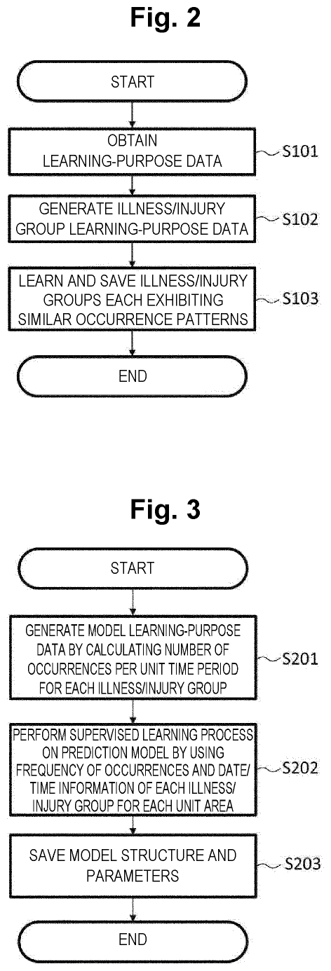

FIG. 2 is a flowchart showing a processing procedure and a processing description of the process of learning the illness/injury groups performed by the emergency medical service demand prediction device 1 shown in FIG. 1.

First, at step S101, under the control of the transport data obtainment unit 21, the emergency medical service demand prediction device 1 obtains the transport data from the external emergency medical service database EMDB via the input/output interface unit 10 and further forwards the obtained transport data to the transport data pre-processing unit 22. At this time, the emergency medical service demand prediction device 1 may save the obtained transport data into the storage unit 30.

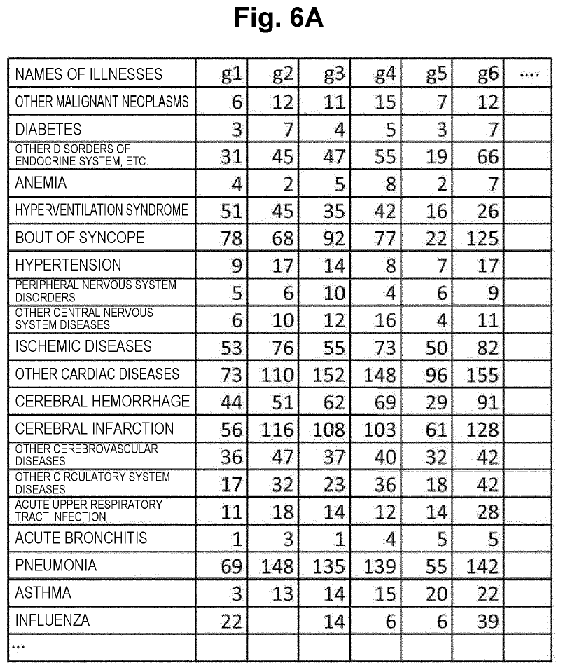

FIG. 5 shows an example of the obtained transport data. The transport data shown in FIG. 5 includes ID numbers identifying records and information indicating dates and times of occurrences, dispatch regions, age groups, genders, places of the occurrences, types of illnesses and injuries, body parts of the illnesses and injuries, degrees of the illnesses and injuries, and the like. The age groups and the genders serve as examples of the patient attributes being the attributes of the patients according to a technique of the present disclosure. The dates and times of occurrences serve as an example of the date/time information indicating the dates and times of the occurrences of the emergency medical service requests according to a technique of the present disclosure. The dispatch regions serve as an example of the position information indicating the places of the occurrences of the emergency medical service requests according to a technique of the present disclosure. The types of the illnesses and injuries, the body parts of the illnesses and injuries, and the degrees of the illnesses and injuries serve as examples of the illness/injury information indicating the illnesses and injuries that caused the emergency medical service requests according to a technique of the present disclosure.

Subsequently, at step S102, under the control of the transport data pre-processing unit 22, the emergency medical service demand prediction device 1 generates illness/injury group learning-purpose data by performing processes such as supplementing missing information or normalization on the transport data received from the transport data obtainment unit 21 and further forwards the generated data to the illness/injury group learning unit 23.

It is considered that illnesses and injuries are impacted by a plurality of factors. For example, there are some situations where the numbers of occurrences of illnesses and injuries may be disproportionately larger in a specific age group or gender. (For example, although many illnesses are often suffered by elderly people, febrile convulsion and the like are often experienced by young people.) Further, there are other situations where the occurrences of illnesses and injuries may be impacted by weather and the like such as temperature or atmospheric pressure or may have regional characteristics (e.g., acute alcoholism in commercial districts, injuries and fractures in sports facilities, and regions having frequent traffic accidents). It is possible to utilize the transport data more efficiently and more effectively, by learning those mechanisms from the data and organizing illness/injury groups that each exhibit similar occurrence patterns in various situations. In the example of the transport data shown in FIG. 5, it is possible to use, as the illness/injury group learning-purpose data, attribute information of the patients such as the age groups and the genders, as well as information about the places of occurrences of the illnesses and injuries, and the dates and times of occurrences of the illnesses and injuries, for example.

Subsequently, at step S103, under the control of the illness/injury group learning unit 23, the emergency medical service demand prediction device 1 learns the illness/injury groups that each exhibit similar occurrence patterns, by using the illness/injury group learning-purpose data and further saves the learning result to the categorization model storage unit 31 as a trained model. To the learning process at the present step, it is acceptable to apply machine learning, a rule determined heuristically, or a calculation of a likelihood based on a certain linear conversion.

For example, the illness/injury group learning unit 23 is capable of calculating degrees of similarity or distances between the illnesses and injuries and extracting the illness/injury groups on the basis of the calculated values.

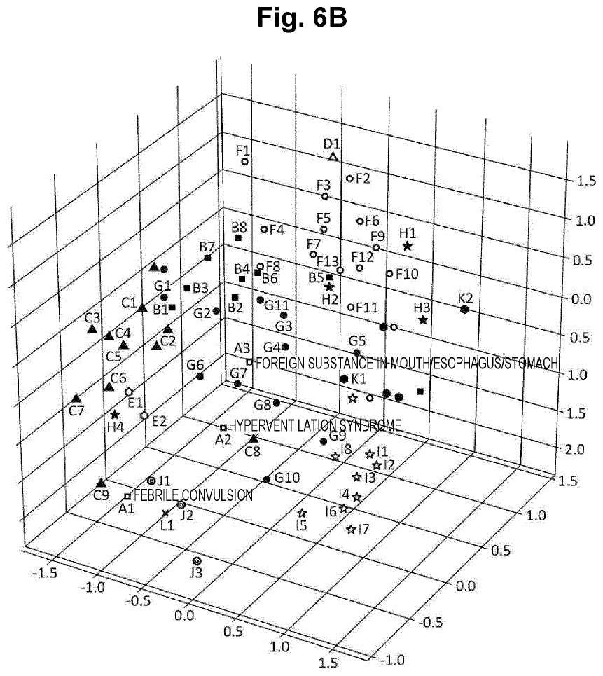

FIG. 6A shows, as an example of the learning process, a result of extracting the illness/injury groups by using a K-means clustering scheme. In the example in FIG. 6A, the illnesses and injuries are divided into tens of clusters, by applying a K-means to data that uses each of the pieces of emergency medical transport data in FIG. 5 as a node and that uses the patient attributes, the places of occurrences, and the dates and times of occurrences as the values of the nodes. These clusters correspond to situations in which illnesses and injuries easily occur. After that, the illnesses and injuries were grouped by performing a clustering process again while using the illnesses and injuries as nodes, and the number of occurrences of illnesses and injuries in each of the clusters as the values thereof. By using the trained model for the illness/injury groups learned by the illness/injury group learning unit 23 in this manner, it is possible to categorize the illnesses and injuries into the illness/injury groups. It is possible to realize the categorization by bringing the patient information, the position information, and the date/time information into association with one another while using the clusters of the illness/injury groups, so as to calculate a likelihood of occurrences of illnesses and injuries with respect to each of the clusters. As for the content of the illness/injury group learning-purpose data that can be used, each piece of data may be used individually or an arbitrary number of pieces of data may be used in combination.

FIG. 6B is a chart further plotting the results of the clustering process in a three-dimensional space by using a multi-dimensional scaling method for visualization. FIG. 6C is a table of a list showing an example in which the results of the clustering process are visualized. The colors and the shapes of the markers express the illness/injury groups to which the illnesses and injuries belong. It is understood, in the present example, that symptoms that are often seen in children and young people such as febrile convulsion and hyperventilation syndrome are organized in the same illness/injury group (white square markers) as symptoms of having a foreign substance in the mouth, esophagus, or stomach that are probably caused by accidental ingestions.

The illness/injury group learning unit 23 is capable of saving the illness/injury groups extracted in this manner into the categorization model storage unit 31 in the form of, for example, a correspondence table between the illnesses and injuries and the illness/injury groups.

Further, the categorization model that has been trained (hereinafter, “trained categorization model”) may be configured so as to perform a re-learning process while using newly-generated learning-purpose data, once every prescribed time period, when a prescribed condition is satisfied, or according to an instruction from the operator or the like.

(2) A Prediction Model Learning Process

The emergency medical service demand prediction device 1 performs a prediction model learning process as described below, upon receipt of an instruction signal to learn the prediction model from the operator, for example, following the illness/injury group learning process or separately from the illness/injury group learning process.

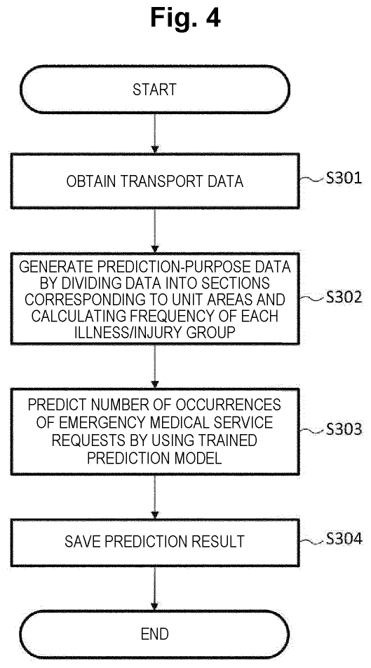

FIG. 3 is a flowchart showing a processing procedure and a processing description of the process of learning the prediction model for predicting the number of occurrences of emergency medical service requests, performed by the emergency medical service demand prediction device 1 shown in FIG. 1.

First, at step S201, on the basis of the transport data obtained by the transport data obtainment unit 21, the emergency medical service demand prediction device 1 generates the prediction model learning-purpose data and further forwards the generated data to the prediction model learning unit 24, under the control of the transport data pre-processing unit 22. The process of generating the prediction model learning-purpose data is performed by reading the trained categorization model and obtaining the number of occurrences per unit time period for each illness/injury group.

For example, at first, the transport data pre-processing unit 22 divides the transport data obtained by the transport data obtainment unit 21 into sections corresponding to unit areas, on the basis of the dispatch region information. In this situation, the unit areas may be municipal districts such as cities, wards, towns, villages, and ‘chome’ levels or may be region meshes calculated on the basis of latitudes and longitudes. It is possible to use an online service such as Google Maps API, for example, for conversions between the municipal districts and the latitudes and longitudes. Subsequently, on the basis of the transport data divided into the sections corresponding to the unit areas, the transport data pre-processing unit 22 performs a process of reading the trained categorization model stored in the categorization model storage unit 31 and further counting, for each unit time period, the number of occurrences of each of the illness/injury groups that have been learned.

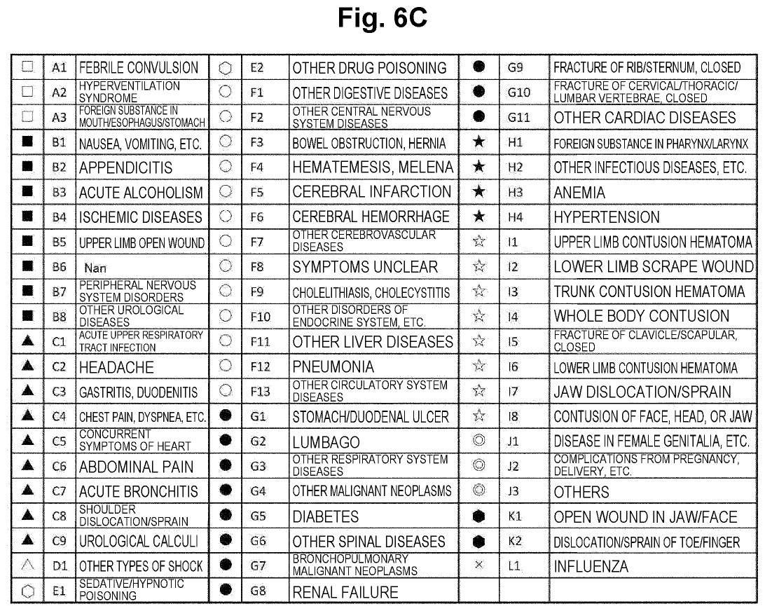

FIG. 7 shows an example of the prediction model learning-purpose data generated in the manner described above. The example in FIG. 7 shows, with respect to “1 Chome, ** Town” serving as a unit area, the number of occurrences of emergency medical service requests for each illness/injury group that is counted for each of the time spans, as well as total numbers of occurrences obtained by adding up the occurrence values.

Subsequently, at step S202, under the control of the prediction model learning unit 24, the emergency medical service demand prediction device 1 performs a supervised learning process on the prediction model by using, as the prediction model learning-purpose data, the frequency of occurrences and the date/time information for each unit area and each illness/injury group as shown in FIG. 7. For the learning process, for example, a statistical method using a generalized linear model or a vector autoregression model, or a machine learning method using a random forest or a neural network may be used.

Subsequently, at step S203, under the control of the prediction model learning unit 24, the emergency medical service demand prediction device 1 saves the trained prediction model, i.e., an optimal model structure and parameters that have been obtained, into the prediction model storage unit 32.

The trained prediction model may be configured so as to perform a re-learning process while using newly-generated learning-purpose data, once every prescribed time period, when a prescribed condition is satisfied, or according to an instruction from the operator or the like.

(3) A Predicting Process

Upon receipt of an instruction signal for the predicting process, the emergency medical service demand prediction device 1 performs a process of predicting the number of occurrences of emergency medical service requests as described below, by using the trained categorization and prediction models.

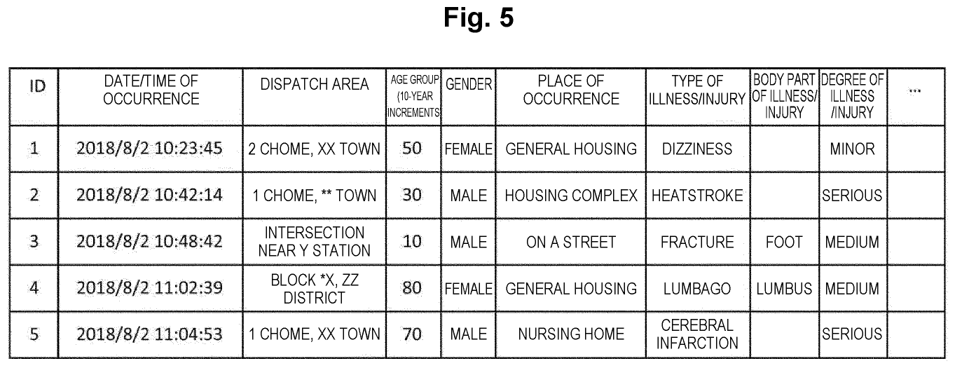

FIG. 4 is a flowchart showing a processing procedure and a processing description of the process of predicting the number of occurrences of emergency medical service requests performed by the emergency medical service demand prediction device 1 shown in FIG. 1.

First, at step S301, under the control of the transport data obtainment unit 21, the emergency medical service demand prediction device 1 obtains the transport data from the external emergency medical service database EMDB via the input/output interface unit 10 and further forwards the obtained transport data to the transport data pre-processing unit 22.

In this situation, generally speaking, because there is a time lag before emergency medical transport data is collected in a database, it is acceptable to use a configuration that takes the time lag into consideration, when the predicting process is performed in actuality. For example, the emergency medical service demand prediction device 1 may be configured so as to obtain the transport data up to a number of hours prior from the emergency medical service database EMDB and to directly collect the most recent transport data from ambulances and the like.

Subsequently, at step S302, under the control of the transport data pre-processing unit 22, the emergency medical service demand prediction device 1 generates prediction-purpose data by performing various types of pre-processing processes on the transport data received from the transport data obtainment unit 21 and further forwards the generated prediction-purpose data to the request occurrence number prediction unit 25. For example, when the most recent transport data is directly obtained from ambulances and the like as described above, the transport data pre-processing unit 22 puts together the transport data obtained from the emergency medical service database EMDB with the most recent transport data, so as to divide the integrated data into the sections corresponding to the unit areas. After that, on the basis of the divided transport data, the transport data pre-processing unit 22 generates the prediction-purpose data to be input to the prediction model, by reading the trained categorization model stored in the categorization model storage unit 31 and further calculating the number of occurrences per unit time period for each illness/injury group. As a result of these processes, the illnesses and injuries are categorized into the illness/injury groups on the basis of the likelihoods of the occurrences of the illnesses and injuries in the illness/injury information obtained by bringing the patient attributes, the position information, and the date/time information into association with one another. Further, frequency of occurrences of the emergency medical service requests is calculated with respect to each of the illness/injury groups, for each prescribed unit area and each prescribed unit time period.

Subsequently, at step S303, under the control of the request occurrence number prediction unit 25, the emergency medical service demand prediction device 1 predicts the number of occurrences of emergency medical service requests at an arbitrary time for each unit area (e.g., for each mesh). The predicting process is performed by reading the trained prediction model stored in the prediction model storage unit 32 on the basis of the received prediction-purpose data. As a result of this process, the number of occurrences of emergency medical service requests is estimated, by bringing the frequency of occurrences of each of the illness/injury groups into association with the arbitrary date/time, while using the prediction model.

At step S304, under the control of the request occurrence number prediction unit 25, the emergency medical service demand prediction device 1 saves the prediction result into the prediction result storage unit 33.

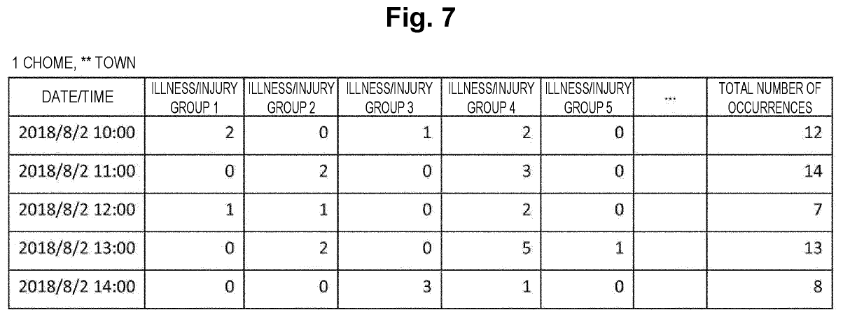

FIG. 8 shows an example of the prediction result obtained in the manner described above. A predicted number of occurrences of emergency medical service requests is indicated for each unit area, the prediction being made for each of the time spans.

The output control unit 26 is capable of reading, at an appropriate time, the prediction result stored in the prediction result storage unit 33, generating output data on the basis of the read prediction result, and outputting the generated output data to a display device or an external device. Alternatively, the request occurrence number prediction unit 25 may be configured to directly forward the prediction result to the output control unit 26. Further, the output control unit 26 is also capable of generating the output data on the basis of the correspondence table stored in the categorization model storage unit 31 or the parameters stored in the prediction model storage unit 32, according to instructions from the operator or the like.

Regarding the learning and predicting processes described above, in an example, the emergency medical service demand prediction device 1 may use “p” sets of information such as the month of occurrence, the days of the week, and holidays, information related to dates and times such as time spans, and the number of occurrences of emergency medical service requests for each type of illnesses and injuries, the p sets corresponding to times tn−m to tn−m+p. For example, the learning process may be configured to perform a supervised learning process to learn a model that predicts a sum of the total numbers of occurrences at times tn to tn+α. For example, when the number of occurrences in the next three hours is to be predicted by using the data from the last 24 hours, the predicting process is able to solve a problem in which a sum of the total numbers of occurrences from times tn to tn+2 is predicted by using learning-purpose data from times tn−24 to tn−1.

According to an aspect of the reference examples, the model learning process may be performed in advance by using the accumulated data, so that only the predicting process is performed during the system operation. Further, an arrangement may be made so that the model re-learning process is performed with prescribed timing (e.g., every week or every month). Alternatively, the re-learning process may be performed when the prediction result has been found significantly incorrect for a prescribed time period.

In one example, the emergency medical service demand prediction device 1 may be configured so that the transport data pre-processing unit 22 inputs actual counted values to the table in the prediction result storage unit shown in FIG. 8 at the stage when the result is confirmed, so that a sum of errors in all the unit areas per unit time period is calculated in order to monitor the sum of errors. Further, when the sum of errors continues to exceed a threshold value, an alarm may be issued to prompt a system operator to run the re-learning process. With this operation method, it is possible to follow changes in the external environment, while keeping costs of the re-learning process down.

### Second Reference Example

In a second reference example, the emergency medical service demand prediction device 1 according to a technique of the present disclosure is further configured to use environment data indicating information about environments of the places of occurrences of the emergency medical service requests, for the learning and predicting processes.

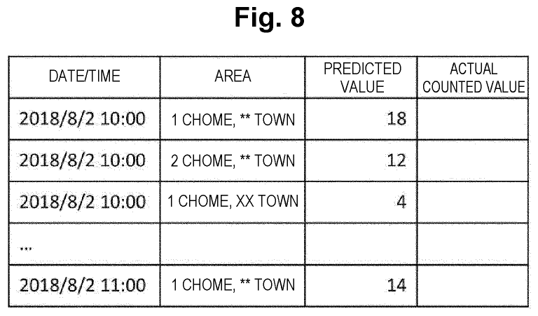

FIG. 9 is a block diagram showing a functional configuration of the emergency medical service demand prediction device 1 according to the second reference example. In FIG. 9, some of the constituent elements that are the same as those in FIG. 1 are referred to by using the same reference characters, and detail explanations thereof will be omitted.

In comparison to the device shown in FIG. 1, the emergency medical service demand prediction device 1 in FIG. 9 further includes, within the control unit 20, an environment data obtainment unit 221 and an environment data pre-processing unit 222. Further, in addition to with the emergency medical service database EMDB, the emergency medical service demand prediction device 1 in FIG. 9 is also capable of communicating with an environment database EVDB via the network NW.

For example, upon receipt of an instruction signal for the learning process or the predicting process input by the operator, the environment data obtainment unit 221 performs a process of obtaining environment data such as meteorological data from the environment database EVDB via the input/output interface unit 10 and further forwarding the obtained data to the environment data pre-processing unit 222. The environment database EVDB is, for example, connected to an environment data collection server that collects information related to the surrounding environment, from the Internet or an external service, either automatically or through manual operations of the operator. The environment database EVDB has accumulated therein the collected environment data.

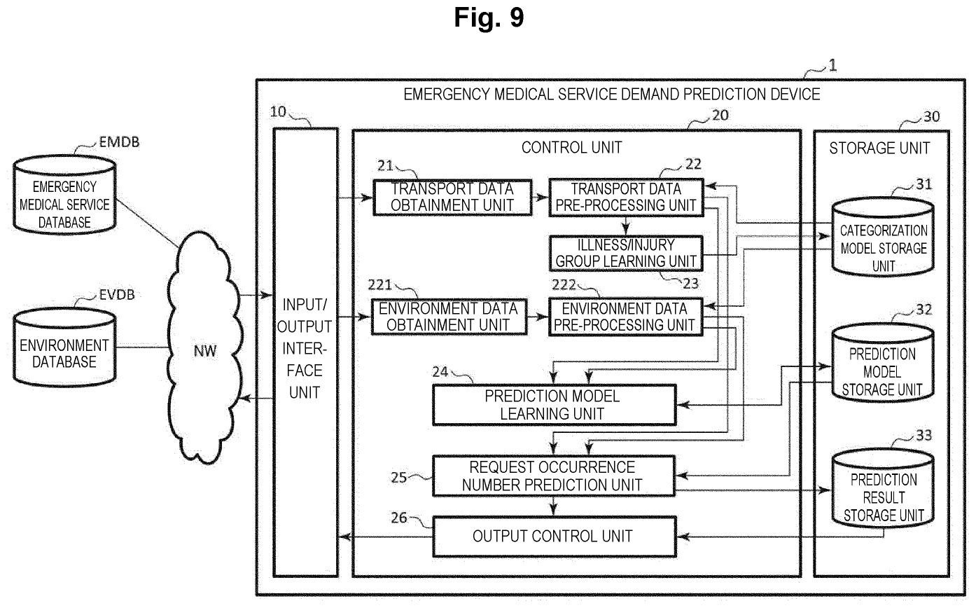

FIG. 10 shows an example of the obtained environment data. An example of the environment data is meteorological data obtained from the homepage of the Japan Meteorological Agency. In FIG. 10, various types of meteorological data are displayed together with the date/time information. Also, information indicating the situations in which the pieces of data were obtained are appended as Conditions 1, 2, and 3. In one example, Condition 1 indicates whether there is any missing data in the data on which the statistics are based. Condition 2 indicates differences in the observation environments. Condition 3 is information indicating whether the corresponding event occurred or not by using the values 0 and 1.

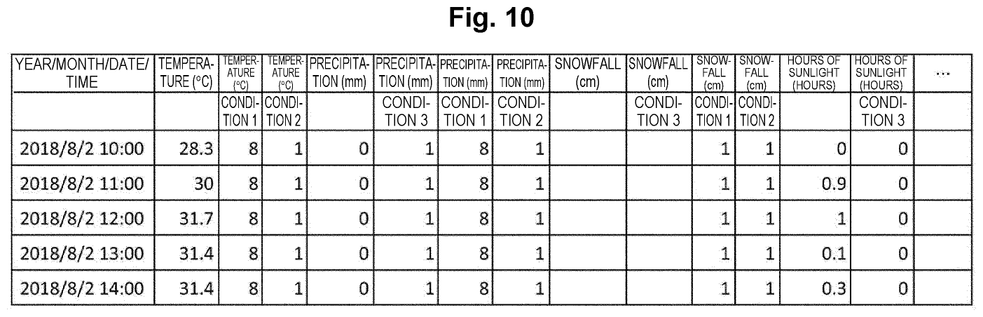

On the environment data received from the environment data obtainment unit 221, the environment data pre-processing unit 222 performs pre-processing processes such as extracting necessary items, supplementing missing information, and performing normalization. FIG. 11 shows an example of the environment data on which the pre-processing processes have been performed. The environment data pre-processing unit 222 forwards the pre-processed environment data to one of the prediction model learning unit 24 and the request occurrence number prediction unit 25.

The prediction model learning unit 24 and the request occurrence number prediction unit 25 are capable of performing the learning and predicting processes, by matching the learning-purpose or prediction-purpose data received from the transport data pre-processing unit 22 with the environment data received from the environment data pre-processing unit 222, on the basis of time information.

### Third Reference Example

In a third reference example, the emergency medical service demand prediction device 1 according to a technique of the present disclosure is further configured to use region data including regional statistic information of the places of occurrences of the emergency medical service requests, for the learning and predicting processes.

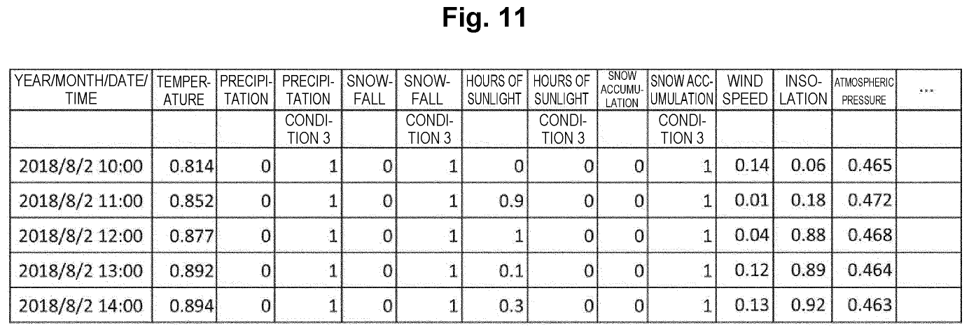

FIG. 12 is a block diagram showing a functional configuration of the emergency medical service demand prediction device 1 according to the third embodiment example. In FIG. 12, some of the constituent elements that are the same as those in FIG. 1 or FIG. 9 are referred to by using the same reference characters, and detail explanations thereof will be omitted.

In comparison to the device shown in FIG. 9, the emergency medical service demand prediction device 1 in FIG. 12 further includes, within the control unit 20, a region data obtainment unit 321 and a region data pre-processing unit 322. Further, in addition to with the emergency medical service database EMDB and the environment database EVDB, the emergency medical service demand prediction device 1 in FIG. 12 is also capable of communicating with a region database via the network NW.

For example, upon receipt of an instruction signal for the learning or predicting process input by the operator, the region data obtainment unit 321 performs a process of obtaining region data from the region database via the input/output interface unit 10 and further forwarding the obtained data to the region data pre-processing unit 322. The region database is, for example, connected to a region data collection server that collects information related to the regional statistics, from the Internet or an external service, either automatically or through manual operations of the operator. The region database has accumulated therein the collected region data. Examples of the regional statistic information contained in the region data include: map information storing facility information of hospitals, shops, and the like in the regions; and information about populations in age groups for each of the unit areas.

On the region data received from the region data obtainment unit 321, the region data pre-processing unit 322 performs region data re-shaping processes such as aggregating data for each unit area, adjusting intervals, and supplementing missing information. The region data pre-processing unit 322 forwards the pre-processed region data to one of the prediction model learning unit 24 and the request occurrence number prediction unit 25.

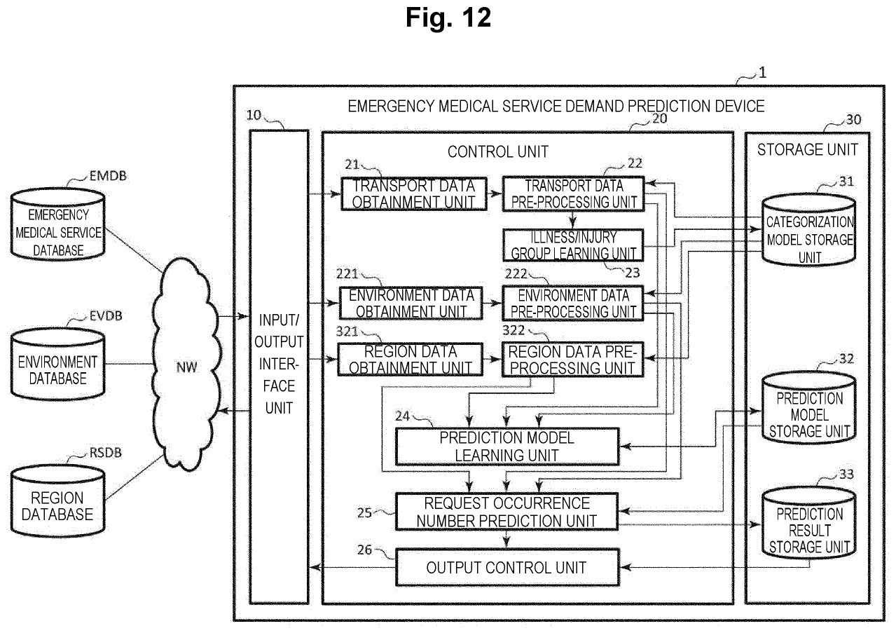

FIG. 13 shows, as an example of the region data, population transition data for different genders and age groups. It is possible to generate the data by, for example, bringing subscriber information into association with terminal device information collected by base stations of mobile phones.

In population distribution data corresponding to different times, the number of people smaller than a threshold value may be masked and shown as blank for the purpose of protecting privacy. In that situation, the region data pre-processing unit 322 is capable of supplementing the missing information in the data, for example, by using a monthly average population recorded as an estimated population or an official registration population prepared by each municipality so as to calculate a value that makes a regional sum of nighttime populations equal to the monthly average population.

The prediction model learning unit 24 and the request occurrence number prediction unit 25 are capable of performing the learning and predicting processes by matching, on the basis of time information, the learning-purpose data or the prediction-purpose data received from the transport data pre-processing unit 22, the environment data received from the environment data pre-processing unit 222, and the region data received from the region data pre-processing unit 322.

Alternatively, it is also acceptable to omit the environment data obtainment unit 221 and the environment data pre-processing unit 222 from the emergency medical service demand prediction device 1 according to the third reference example, so as not to use the environment data in the learning and predicting processes.

### Fourth Reference Example

In a fourth reference example, the emergency medical service demand prediction device 1 according to a technique of the present disclosure is further configured to learn regional characteristics of each unit area on the basis of the region data indicating the regional statistic information of the places of occurrences of the emergency medical service requests and to use a learning result for the learning and predicting processes described above.

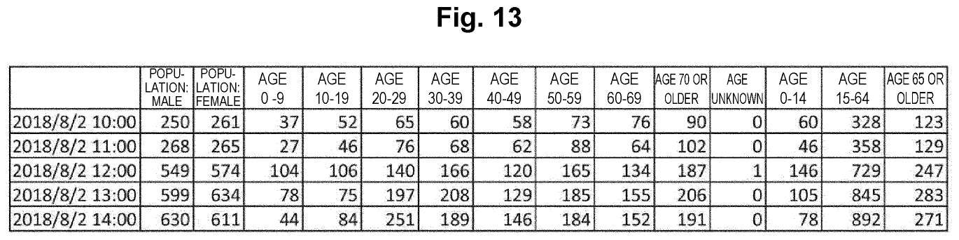

FIG. 14 is a block diagram showing a functional configuration of the emergency medical service demand prediction device 1 according to the fourth reference example. In FIG. 14, some of the constituent elements that are the same as those in FIG. 1, 9, or 12 are referred to by using the same reference characters, and detail explanations thereof will be omitted.

In comparison to the device shown in FIG. 12, the emergency medical service demand prediction device 1 in FIG. 14 further includes a regional characteristic learning unit 421 within the control unit 20 and includes a regional characteristic storage unit 434 within the storage unit 30.

Similarly to the third reference example, the emergency medical service demand prediction device 1 shown in FIG. 14 obtains the region data under the control of the region data obtainment unit 321 and performs a prescribed pre-processing process on the obtained region data under the control of the region data pre-processing unit 322. The region data pre-processing unit 322 forwards the pre-processed region data to one of the prediction model learning unit 24, the request occurrence number prediction unit 25, and the regional characteristic learning unit 421. For example, on the basis of the obtained region data, the region data pre-processing unit 322 is capable of performing one of the following: generating learning-purpose region data to forward the generated data to the prediction model learning unit 24; generating prediction-purpose region data to forward the generated data to the request occurrence number prediction unit 25; and generating regional characteristic learning-purpose data to forward the generated data to the regional characteristic learning unit 421.

While using the received regional characteristic learning-purpose data, the regional characteristic learning unit 421 performs a process of learning and extracting information indicating what type of district the region is, on the basis of the population data for each time span or each age group, as well as the numbers of hospitals, nursing homes, shops, schools, sports facilities, and the like in the areas.

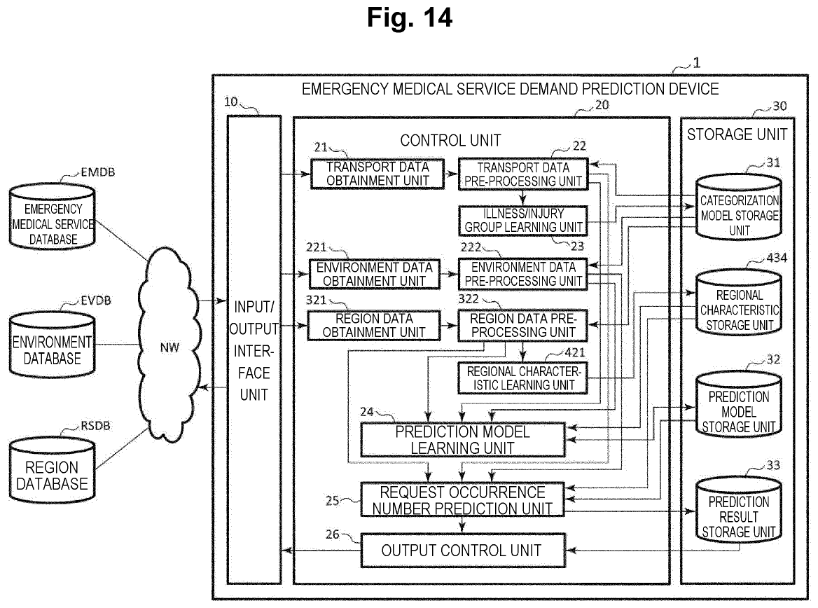

FIG. 15 shows an example of the regional characteristic learning-purpose data. It is possible to obtain the types and the numbers of facilities in the areas, by conducting searches in various types of map services such as Google Maps API.

The regional characteristic learning unit 421 does not function when the learning process is performed on a single unit area, but becomes able to extract, as a learning result, feature values expressing regional characteristics, by performing a learning process on various types of unit areas and having the parameters thereof handed over. By using the feature values expressing the regional characteristics, it is possible to extract an emergency medical service request occurrence pattern corresponding to the nature of each district such as a business district or a commercial district and the nature of residents such as a mature residential area or a newly-developed residential area.

The regional characteristic storage unit 434 stores therein, as the learning result, the feature values expressing the regional characteristics obtained by the regional characteristic learning unit 421. Similarly to the storage units 31, 32, and 33, the regional characteristic storage unit 434 is not a requisite configuration and may be replaced by an external storage medium or the like.

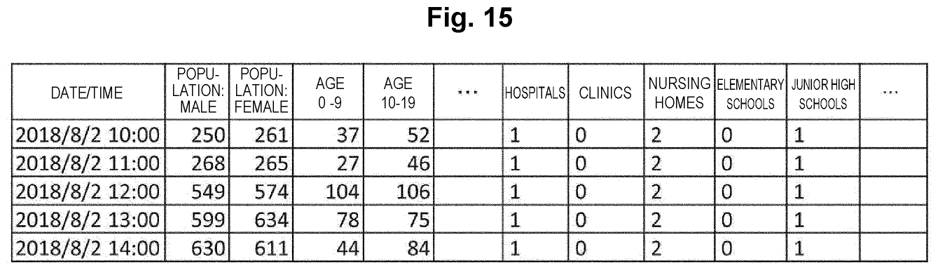

FIG. 16 shows, as an example of the learning result obtained by the regional characteristic learning unit 421, a result of conjecturing which nature each of the areas may have such as the nature of a business district, a commercial district, a residential area, or the like, on the basis of population fluctuations and the facilities that are present in the area.

The prediction model learning unit 24 and the request occurrence number prediction unit 25 are capable of performing the learning and predicting processes described above, on the basis of various types of data. The various types of data include: the learning-purpose data or the prediction-purpose data received from the transport data pre-processing unit 22; the environment data received from the environment data pre-processing unit 222; the region data pre-processed by the region data pre-processing unit 322; and the feature values expressing the regional characteristics and having been extracted by the regional characteristic learning unit 421.

Alternatively, it is also acceptable to omit the environment data obtainment unit 221 and the environment data pre-processing unit 222 from the emergency medical service demand prediction device 1 according to the fourth reference example, so as not to use the environment data in the learning and predicting processes.

### Fifth Reference Example

The emergency medical service demand prediction device 1 in the first reference example independently extracts the illness/injury groups by using the clustering scheme; however, the illness/injury group extracting process and the prediction model learning process may be performed simultaneously.

In a fifth reference example, the emergency medical service demand prediction device 1 according to a technique of the present disclosure simultaneously performs the illness/injury group extracting process and the prediction model learning process, by adding a layer corresponding to the illness/injury group learning process to the model in a neural network, for example.

In the fifth reference example, for instance, a Long Short-term Memory [LSTM] (a type of recurrent neural network) layer is prepared for the illness/injury group extracting process and for the occurrence number predicting process. With the LSTM layer for the illness/injury group extracting process, it is possible to set, as an input, a count of the number of occurrences of emergency medical service requests per unit time period, for each of various types of illnesses and injuries determined by each municipality. Further, by setting the number of output nodes to a small value (approximately 20 to 30) relative to the original number of illness/injury categories, it is possible to achieve an advantageous effect where the illness/injury categories are summarized. By setting the output as an input to the LSTM layer for the emergency medical service request occurrence number predicting process and proceeding with the learning process collectively, it is possible to extract illness/injury groups that improve the level of precision of the occurrence number prediction to a maximum extent.

(1) The Learning Process

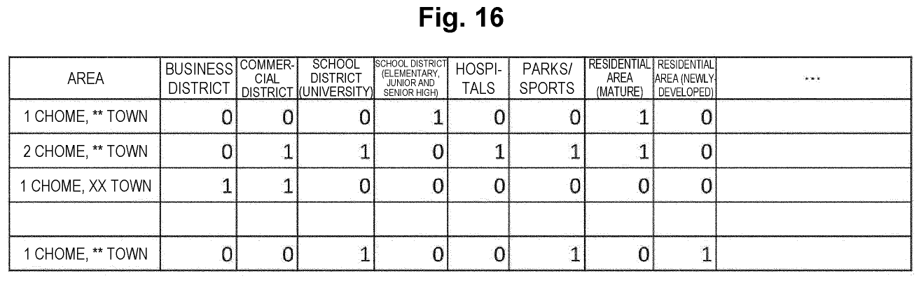

FIG. 17 is a flowchart showing the processes of learning the illness/injury groups and the prediction model performed by the emergency medical service demand prediction device 1 according to the fifth reference example. The basic configuration of the emergency medical service demand prediction device 1 according to the fifth reference example is the same as that of the emergency medical service demand prediction device 1 shown in FIG. 1, except how the functions are executed is different. Thus, in the following sections, processes will be explained by using the reference characters of the constituent elements of the emergency medical service demand prediction device 1 shown in FIG. 1.

First, at step S401, under the control of the transport data obtainment unit 21, the emergency medical service demand prediction device 1 obtains the transport data from the external emergency medical service database EMDB via the input/output interface unit 10 and further forwards the obtained transport data to the transport data pre-processing unit 22.

Subsequently, at step S402, under the control of the transport data pre-processing unit 22, the emergency medical service demand prediction device 1 generates model learning-purpose data. The model learning-purpose data is generated by dividing the transport data received from the transport data obtainment unit 21 into sections corresponding to the unit areas and further counting the number of occurrences per unit time period for each of the illnesses and injuries based on the categorization of each municipality.

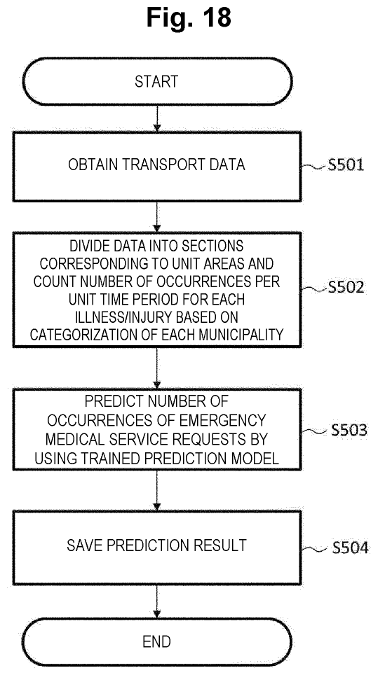

FIG. 19 shows an example of the model learning-purpose data. With the LSTM layer for the illness/injury group extracting process, it is possible to set, as an input, model learning-purpose data obtained by counting the number of occurrences of emergency medical service requests per unit time period, for each of the various types of illnesses and injuries determined by each municipality, as shown in FIG. 19.

Subsequently, at step S403, the emergency medical service demand prediction device 1 performs the illness/injury group learning process by the illness/injury group learning unit 23, together with the prediction model learning process by the prediction model learning unit 24.

At step S404, the emergency medical service demand prediction device 1 saves a model structure and parameters related to the trained prediction model into the prediction model storage unit 32, for example.

(2) A Predicting Process

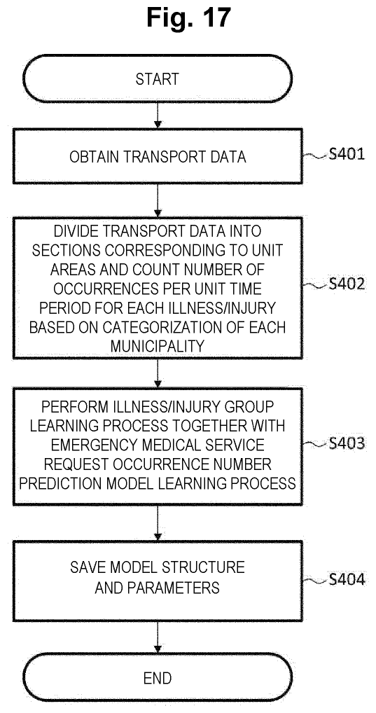

FIG. 18 is a flowchart showing the predicting process performed by the emergency medical service demand prediction device 1 according to the fifth reference example. Again, processes will be explained by using the reference characters of the constituent elements of the emergency medical service demand prediction device 1 shown in FIG. 1.

First, at step S501, under the control of the transport data obtainment unit 21, the emergency medical service demand prediction device 1 obtains the transport data from the external emergency medical service database EMDB via the input/output interface unit 10 and further forwards the obtained transport data to the transport data pre-processing unit 22. As explained in the first reference example, in the actual predicting process, the emergency medical service demand prediction device 1 may directly collect the most recent transport data from ambulances and the like, in consideration of the time lag before certain emergency medical transport data is collected into the database.

Subsequently, at step S502, under the control of the transport data pre-processing unit 22, the emergency medical service demand prediction device 1 divides the transport data received from the transport data obtainment unit 21 into sections corresponding to the unit areas and further generates prediction-purpose data by counting the number of occurrences per unit time period for each of the illnesses and injuries based on the categorization of each municipality.

At step S503, under the control of the request occurrence number prediction unit 25, while using the trained prediction model that has been learned as described above, the emergency medical service demand prediction device 1 predicts the number of occurrences of emergency medical service requests for each unit area on the basis of the prediction-purpose data.

At step S504, under the control of the control unit 20, the emergency medical service demand prediction device 1 saves the prediction result obtained by the request occurrence number prediction unit 25, into the prediction result storage unit 33.

Further, similarly to the illness/injury group extracting process, the emergency medical service demand prediction device 1 may also perform the regional characteristic learning process described in the fourth reference example simultaneously together with the prediction model learning process. In that situation, for example, by incorporating the regional characteristic extracting process and the emergency medical service request occurrence number predicting process into mutually the same model by using a neural network, it is possible to optimize both at the same time. More specifically, for example, the regional characteristic learning unit 421 may be configured as an LSTM layer that receives an input of a distribution of population for each age group corresponding to the days of the week and time spans and the facility information in the areas and outputs approximately 10 to 20 nodes corresponding to different types of districts.

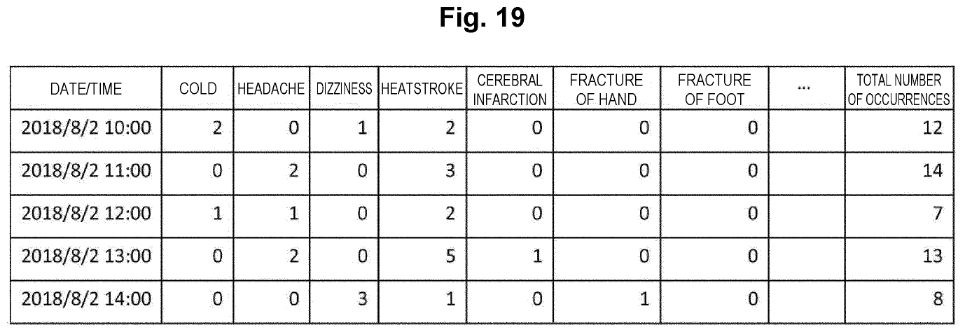

FIG. 20 shows an outline of data flows in the learning model using the LSTM layer according to the fifth reference example described above. The emergency medical service demand prediction device 1 shown in FIG. 20 uses an LSTM layer that receives an input of the data indicating the number of occurrences for each type of illnesses and injuries obtained from each municipality for extracting the illness/injury groups and further uses the output of the LSTM layer as an input to a prediction-purpose LSTM. Similarly, the emergency medical service demand prediction device 1 uses an LSTM layer for extracting regional characteristic feature values and further uses the output of the LSTM layer as an input to a prediction-purpose LSTM.

### Sixth Reference Example

In a sixth reference example, the emergency medical service demand prediction device 1 according to a method of the present disclosure is further configured so that the output control unit 26 generates and outputs output data for visually displaying the prediction result. The emergency medical service demand prediction device 1 according to the sixth reference example may have the same functional configuration as that of any of the emergency medical service demand prediction devices 1 described in the first to the fifth reference examples.

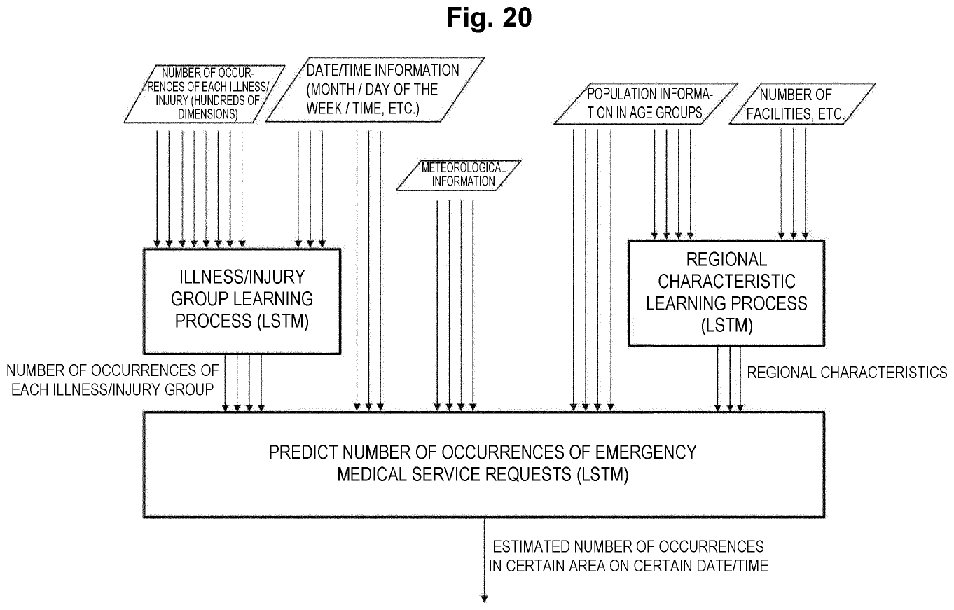

FIGS. 21A and 21B show examples of the display based on the output data output from the emergency medical service demand prediction device 1 according to the sixth reference example in which prediction results are visualized on heat maps. These images may be displayed on a display device such as a liquid crystal display device.

FIG. 21A shows an emergency medical service demand prediction result for three hours later, predicted on the basis of the most recent data. FIG. 21B shows an emergency medical service demand prediction result for six hours later, predicted on the basis of the most recent data. From FIGS. 21A and 21B, it is observed that, between the predictions for the three hours later and for the six hours later, the area predicted with a larger number of occurrences of emergency medical service requests (the darker meshes in the heat maps) has shifted from the lower left corner to the upper right corner. The emergency medical service demand prediction device 1 according to the sixth reference example is also capable of assisting exploring the utilization of emergency medical staff, by depicting transitions of predicted values in a specific area in a graph or a table. Further, the output format of the prediction result from the emergency medical service demand prediction device 1 according to the sixth reference example is not limited to visual presentation. It is possible to output the result in various formats including a synthetic voice.

### Advantageous Effects of Reference Examples

As explained in detail above, the emergency medical service demand prediction device according to the one reference example is capable, in emergency medicine, of predicting the number of occurrences of emergency medical service requests in the near future, from the transport data including the dates and times of occurrences and the places of occurrences of the emergency medical service requests from the past. At that time, the past emergency medical transport data is divided into the pieces of data corresponding to the unit areas, so that the number of occurrences is calculated in each area for each unit time period and each type of illnesses and injuries as a feature value. After the feature values are calculated, the illness/injury groups that are each impacted by the same type of environment factors are learned, so as to perform the learning and predicting processes by using the number of occurrences calculated for each illness/injury group that has been learned, as the learning-purpose data and the prediction-purpose data.

With these arrangements, even when the obtained actual history data of the emergency medical transports do not have sufficiently high frequency of occurrences that can withstand analyses in units of illnesses and injuries, it is possible to predict the number of occurrences of the emergency medical service requests with an excellent level of precision from the limited actual history data, by learning the illness/injury groups and calculating the frequency of occurrences for each illness/injury group to be used in the analyses.

Further, as described above, there are certain illnesses and injuries of which the numbers of occurrences are disproportionately larger in a specific age group or gender. Further, there are certain illnesses and injuries of which the occurrences are impacted by temperature, atmospheric pressure, or weather or have regional characteristics. For these reasons, illnesses and injuries are considered to be impacted by a plurality of factors. The emergency medical service demand prediction device according to the one reference example learns the mechanism of these factors from the data and organizes the illness/injury groups that each exhibit the similar occurrence patterns in various situations. It is therefore possible to absorb differences in the manual categorization and to handle the data of the number of occurrences of the emergency medical service requests for each type of illnesses and injuries, with optimal granularity.

Further, generally speaking, many illnesses are impacted by temperature or weather, (e.g., a cold, the influenza, heatstroke, etc.). Further, changes in the atmospheric pressure are known to disturb the autonomic nerves and cause various illnesses such as headaches, nerve pains, and strokes. The emergency medical service demand prediction device according to the one reference example incorporates these impacts in the models by performing the learning and predicting processes while using the environment data such as the meteorological data. It is thus expected to be possible to achieve an advantageous effect where the levels of precision of the predictions are improved.

Further, typified by the fact that newly-developed residential areas attract families raising children, residential areas often attract residents who belong to similar age groups or who have similar financial statuses, values, and/or lifestyles. Further, requests for emergency medical service dispatches strongly reflect regional characteristics in that, for example, regions having a hospital experience a certain ratio of emergency medical service dispatches for inter-hospital transports, and in commercial districts, the number of requests for an emergency medical service dispatch increases during nighttime for acute alcoholism. The emergency medical service demand prediction device according to the one reference example is able to incorporate the impacts of these factors in the models, by performing the learning and predicting processes that utilize the regional characteristics and is thus able to improve the levels of precision of the predictions.

Further, generating and outputting the output data by using a heat map or the like so as to visualize the prediction result makes it possible to understand the prediction result easily and efficiently in the actual emergency medical situations where prompt reactions are required. Further, by using a graph or the like, it is also possible to easily visualize chronological transitions in the number of occurrences of emergency medical service requests. Consequently, it is possible to visualize an estimated number of dispatches of emergency medical staff predicted for a number of hours later and to thus improve the efficiency in utilization of the emergency medical staff.

As explained above, the emergency medical service demand prediction device according to the one reference example is able to predict the number of occurrences of emergency medical service requests with an excellent level of precision from the limited observation data, by extracting the illness/injury groups that each have similar occurrence patterns from the past emergency medical transport data and performing the learning process on the basis of the number of occurrences of each illness/injury group. Further, the emergency medical service demand prediction device according to the one reference example is able to estimate the number of occurrences of emergency medical service requests in each area with a high level of precision by using various types of data in combination, such as the emergency medical transport data collected from the control offices and the emergency medical staff, the meteorological data that is available online, the regional characteristics of each of the unit areas, and the distribution of population per unit time period by the age groups. Further, it is possible to absorb the differences among the municipalities in the categorizations of the illnesses and injuries in the emergency medical transport data and to thus easily construct the models that are usable across the plurality of municipalities.

### Other Reference Examples

Possible methods of the reference examples are not limited to the reference example 1 described above. For example, the trained model and the parameters saved in the prediction model storage unit 32 may conform to parameter-saving processes and file formats compliant with a statistical analysis tool being used. Similarly, the formats of the data saved in the categorization model storage unit 31, the prediction result storage unit 33, and the regional characteristic storage unit 434 are not limited to the examples presented in the drawings. It is acceptable to use arbitrary formats.

Further, possible methods for the illness/injury group learning process and the prediction model learning process are not limited to the statistical method and the machine learning method described above. It is acceptable to use arbitrary methods.

In the above example, the environment data including the meteorological information and the region data including the facility information and the population information are used for the learning and predicting processes of the prediction model; however, it is acceptable to also use these pieces of data for the illness/injury group learning process. In particular, in the reference examples in which the categorization model learning process is performed separately from the prediction model learning process, it is expected that it is possible to perform the illness/injury group learning process more efficiently, by using the environment data and the region data.

Further, it is possible to combine, to replace with a similar element, or to omit any of the functional units of the emergency medical service demand prediction device 1 described in the first to the sixth reference examples. For example, as described earlier, it is possible to configure the third and the fourth reference examples so that the learning and predicting processes are performed on the basis of the transport data and the region data without using the environment data.

Alternatively, the functional units 21 to 26 included in the emergency medical service demand prediction device 1 may be provided in a cloud computer, an edge router, or the like in a distributed manner, so that the learning and predicting processes are performed as a result of these devices collaborating with one another. With this arrangement, it is possible to reduce the processing loads of the devices and to thus improve the efficiency of the processes.

According to an aspect of the reference examples described above, the prediction model is constructed by using the deep learning or the like so as to predict the number of occurrences of the emergency medical services in a certain area, by using the emergency medical service dispatch data from the past, the meteorological data, the dynamic population data, and the region information. In particular, an aspect of the reference examples is characterized with extracting the illness/injury groups exhibiting a similar emergency medical service occurrence pattern, so as to make a prediction for each of the illness/injury groups. One prediction model is generated for each unit area.

### Embodiment Examples

In view of the reference examples described above, a method according to an embodiment of the present disclosure proposes an ensemble model obtained by separately constructing a prediction model that learns outbursts of increases and decreases that occur for each of the illnesses and injuries or for each of the illness/injury groups and further preparing combinations by using meteorological condition and date/time information.

In the ensemble model, for example, a focus is placed on the following points: [1] Even for a time period exhibiting a constant tendency, a learning process is performed while applying weights so as to obtain a time-series model taking into consideration that tendencies are different between the spring/fall seasons and the summer/winter seasons. Also, [2] A learning process is performed while applying weights, so as to obtain an outburst model taking into consideration a tendency where the number of requests increases with characteristics as follows: The number of requests increases after a prescribed time period has elapsed since a trigger being an event (e.g., a sudden rise or drop in temperature, snow accumulation, and custom such as the New Year holidays) including a certain element that, upon occurrence, increases the number of requests without being dependent on time-series changes. In the ensemble model of the present embodiment, the weights are applied while taking [1] and [2] into consideration. Further, as for situations where the number of occurrences increases once and is maintained for a specific time period, weights may be applied on the assumption that these situations are included in [1].

In one configuration example of the ensemble model according to the embodiment of the present disclosure, the following two prediction models are learned by using mutually-different methods, so as to construct a prediction model that outputs a weighted sum thereof. The first one is a prediction model that primarily learns time-series periodicity or trends corresponding to normal times. The second one is a prediction model that learns outbursts of increases caused by drastic meteorological changes that are frequently observed in the summer and the winter. As for the learning method of each of the prediction models, for example, the prediction model at normal times may adopt a vector autoregression model that performs a time-series analysis for each of the illness/injury groups. In contrast, the prediction model for outburst events used for grasping changes caused by the outburst events may adopt a deep learning model, which is excellent in pattern recognitions. As explained herein, the prediction model at normal times grasps constant changes, whereas the prediction model for outburst events grasps non-constant changes.

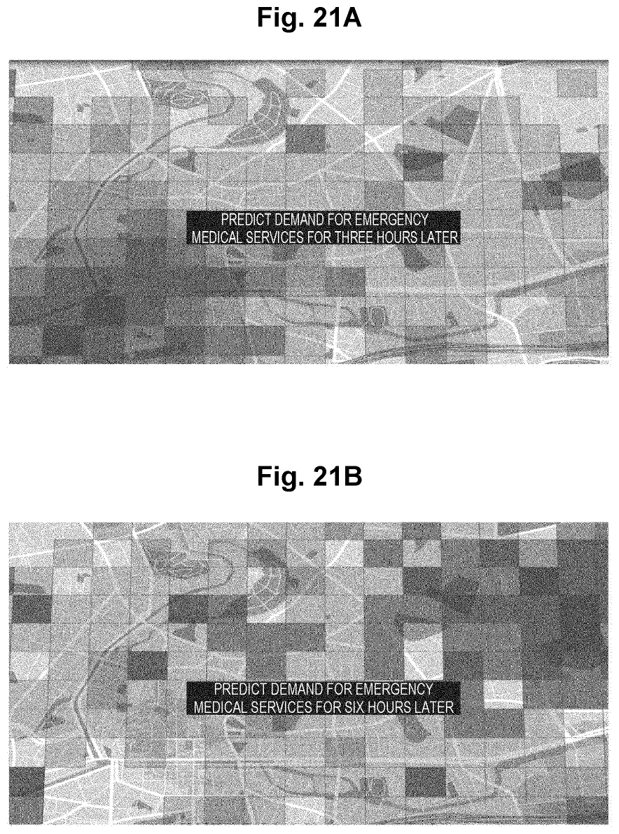

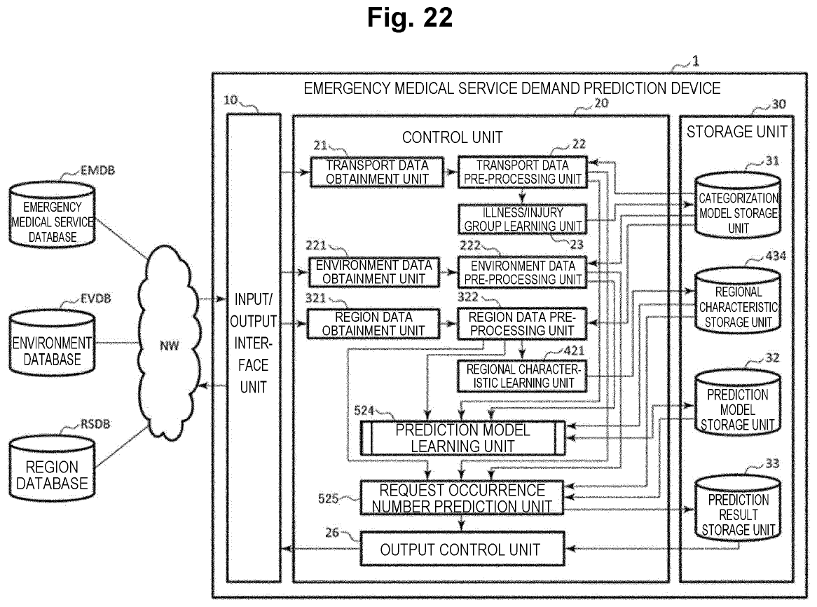

FIG. 22 is a drawing showing examples of prediction results obtained when a vector regression model is used as a prediction model. FIG. 23 is a drawing showing examples of prediction results obtained when a deep learning model is used as a prediction model. FIG. 22 and FIG. 23 present graphs indicating the results of predicting the number of emergency medical service dispatches in every three-hour period in a certain city in 2017, by using a prediction model trained with data from the years 2013 to 2016. In FIG. 22 and FIG. 23, each of the three months of May, June, and July is shown in the graph in which the solid line indicates the actual number of dispatches, whereas the dashed line and the broken line show the predicted values. The mean square error throughout the year was 5.78 for the vector autoregression model and was 7.79 for the deep learning model. Thus, the vector autoregression exhibited a higher precision level of prediction. Further, as for the prediction of heatstroke in the second half of July, it is observed that the deep learning model is able to follow the peaks from the first day onward.

Further, when the plurality of prediction models are combined together, the number of occurrences of the emergency medical services is significantly impacted not only by the date/time information, but also by meteorological situations. Accordingly, a method according to an embodiment of the present disclosure proposes an ensemble model that controls the weights by using two types of information, namely, the date/time information and the meteorological condition.

Conventionally, there have not been sufficient studies on methods for combining mutually-different prediction models into an ensemble. Known methods are limited to those by which an analysis result is simply determined by majority and by which weights are determined on the basis of the precision level of each model. To cope with this situation, according to the method in the embodiment of the present disclosure, an appropriate model ensemble is constructed by specializing in emergency medical service areas. In other words, the meteorological information and the date/time information, which are the causes of the changes in the number of emergency medical service dispatches, are used as attributes, optimal weights corresponding to changes in meteorological situations and dates/times are learned through machine learning.

<A Configuration>

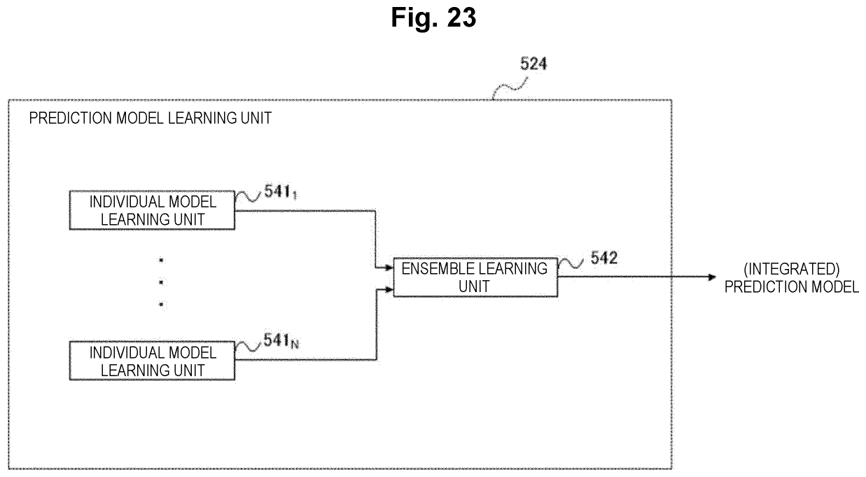

FIG. 24 is a block diagram showing an example of a functional configuration of an emergency medical service demand prediction device according to one embodiment of the present disclosure. Some of the constituent elements that are the same as those in the reference examples are referred to by using the same reference characters, and detail explanations thereof will be omitted. Primary configurations of the embodiment of the present disclosure shown in FIG. 24 lie in aspects of a prediction model learning unit 524 and a request occurrence number prediction unit 525. The emergency medical service demand prediction device is an example of the information processing device according to a technique of the present disclosure.

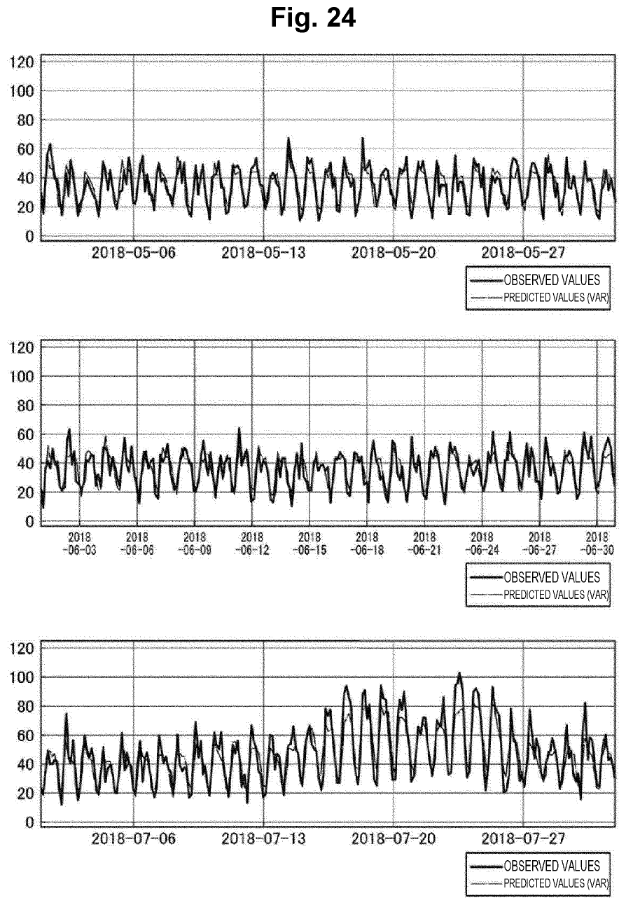

At first, a learning process of the prediction model learning unit 524 will be explained. FIG. 25 is a block diagram showing a detailed configuration of the prediction model learning unit 524 according to one embodiment of the present disclosure. As shown in FIG. 25, the prediction model learning unit 524 is configured so as to include individual model learning units 541N (5411 to 541N) and an ensemble learning unit 542.

The prediction model learning unit 524 receives learning-purpose data and the like, learns individual prediction models by employing each of the individual model learning units 541N, and learns an integrated prediction model integrating together the individual prediction models by employing the ensemble learning unit 542. Similarly to the reference examples described above, the integrated prediction model is a prediction model used for predicting a total number of occurrences of emergency medical service requests on the basis of the number of occurrences of each of the illness/injury groups and the date/time information.

A learning process of the individual model learning units 541N will be explained. The letter “N” of the individual model learning units 541N corresponds to the number of individual prediction models. The number of individual prediction models is set in advance so that, as explained above, it is possible to make the prediction at normal times and the prediction of the outburst events. For example, when learning the prediction model at normal times and the prediction model for the outburst events, the letter “N” of the individual model learning units 541N set to satisfy N=2, so that each of the prediction models is learned by assigning 1 to the normal times and 2 to the outburst events. Alternatively, instead of putting the outburst events in the summer and the winter together, it is also acceptable to apply N=3 so as to have a prediction model for outburst events in the summer and a prediction model for outburst events in the winter.

To learn the prediction model at normal times, a learning process is performed by using a vector regression model. To learn the prediction model for the outburst events, a learning process is performed by using a deep learning model. In both of the learning processes, the individual model learning units 541N perform the learning processes by receiving inputs of various types of learning-purpose data. The various types of learning-purpose data include, for example, the learning-purpose data from the transport data pre-processing unit 22, the environment data from the environment data pre-processing unit 222, the region data from the region data pre-processing unit 322, and the feature values from the regional characteristic learning unit 421 expressing the regional characteristics. The date/time information in the learning-purpose data includes information about the days of the week, non-business days, holidays, and the like. In the present example, the prediction model at normal times is an example of the first method according to a technique of the present disclosure. The prediction model for the outburst events is an example of the second method according to a technique of the present disclosure. As explained above, the prediction model for the outburst events exhibits a higher precision level in the pattern recognition of sections having the occurrences of outburst events in comparison to the prediction model at normal times. By using the second method, increases and decreases in the number of occurrences of emergency medical service requests being different from time-series fluctuation are estimated. In this situation, the time-series fluctuation denotes, for example, fluctuation in the number of occurrences estimated by the prediction model at normal times.

A learning process of the ensemble learning unit 542 will be explained. The ensemble learning unit 542 receives inputs of: the outputs of the individual prediction models included in the individual model learning units 541N; the emergency medical transport data including a training signal; and the meteorological condition and the date/time information serving as indices of weights used for determining an output. The abovementioned prediction-purpose data is used as the emergency medical transport data and the date/time information. The abovementioned environment data is used as the meteorological data. While using these pieces of input data as an input to a neural network, the ensemble learning unit 542 performs an ensemble learning process in which weights on the neural network are varied in accordance with the seasons, the days of the week, time spans of the day, and meteorological situations at different times, so as to obtain and save the integrated prediction model into the prediction model storage unit 32. The integrated prediction model is trained so as to adjust the increases and decreases in the number of occurrences of the emergency medical service requests by using the date/time information and the meteorological condition as the weights. As for the date/time information, the weights are adjusted on the basis of information about the months, the time spans of the day, the days of the week, and whether the days are each a non-business day or not, and the like. As for the meteorological condition, the weights are adjusted on the basis of information about temperatures (e.g., a difference from an annual average temperature or a temperature difference from the previous day) and weather. For example, the weights may be learned in such a manner that the more it snows or it rains, the larger is the weight, i.e., the larger is the number of occurrences of the emergency medical service requests. The integrated prediction model also includes the individual prediction models learned by the individual model learning units 541N.

Next, a predicting process of the request occurrence number prediction unit 525 will be explained. As explained in the reference examples, as a process at a stage preceding the predicting process, the transport data pre-processing unit 22 generates the various types of prediction-purpose data to be input to the prediction model. The various types of prediction-purpose data are generated by reading the trained categorization model stored in the categorization model storage unit 31 on the basis of the divided transport data and further calculating the number of occurrences per unit time period for each of the illness/injury groups. As a result, the illnesses and injuries are categorized into the illness/injury groups, so that frequency of occurrences of each of the illness/injury groups is calculated for each prescribed unit area and each prescribed unit time period. Further, the various types of prediction-purpose data include the environment data from the environment data pre-processing unit 222, the region data from the region data pre-processing unit 322, and the feature values from the regional characteristic learning unit 421 expressing the regional characteristics. The transport data pre-processing unit 22 is an example of the pre-processing unit according to a technique of the present disclosure.

The request occurrence number prediction unit 525 performs the predicting process by using the various types of prediction-purpose data and the integrated prediction model stored in the prediction model storage unit 32. In the predicting process using the integrated prediction model, for example, the number of occurrences of the emergency medical service requests is estimated by using each of the individual prediction models included in the integrated prediction model, and subsequently, the number of occurrences of the emergency medical service requests to be output eventually is obtained on the basis of each of the numbers of occurrences and the weights. The request occurrence number prediction unit 525 is an example of the prediction unit according to a technique of the present disclosure.

In the predicting process using the prediction model at normal times, the number of occurrences of the emergency medical service requests is estimated by bringing an arbitrary date/time into association with the frequency of occurrences of each of the illness/injury groups calculated for each prescribed unit area and each prescribed unit time period, while using the prediction model at normal times. Similarly, in the predicting process using the prediction model for the outburst events, increases and decreases in the number of occurrences of the emergency medical service requests are estimated by bringing an arbitrary date/time into association with the frequency of occurrences of each of the illness/injury groups calculated for each prescribed unit area and each prescribed unit time period, while using the prediction model for the outburst events. The increases and decreases in the number of occurrences may be calculated, for example, by making a comparison with the number of occurrences estimated by using the prediction model at normal times. Examples of the predictions using each of the prediction models are presented in FIG. 22 and FIG. 23 explained above.

The request occurrence number prediction unit 525 obtains the number of occurrences of the emergency medical service requests to be output, by using the integrated prediction model and saves the obtained result into the prediction result storage unit 33. The number of occurrences of the emergency medical service requests to be output is obtained on the basis of the number of occurrences estimated by using the prediction model at normal times, the increases and decreases in the number of occurrences estimated by using the prediction model for the outburst events, the arbitrary date/time, and the weights obtained on the basis of at least the meteorological condition. As the date/time information and the meteorological condition, information from the prediction data may be used as appropriate. As for the meteorological condition, the temperature, the weather, and the like corresponding to the position information may be obtained from the environment data. When the numbers of occurrences, the date/time information, and the meteorological condition are input to the neural network of the integrated prediction model, the weights on the neural network are calculated so that the neural network outputs the number of occurrences of the emergency medical service requests for each of the illness/injury groups corresponding to the weights.

<Operations>

Operations in a learning process and a predicting process will be explained. The learning process and the predicting process are realized under the control of functional units as a result of causing a processor to execute a program stored in the storage unit 30.

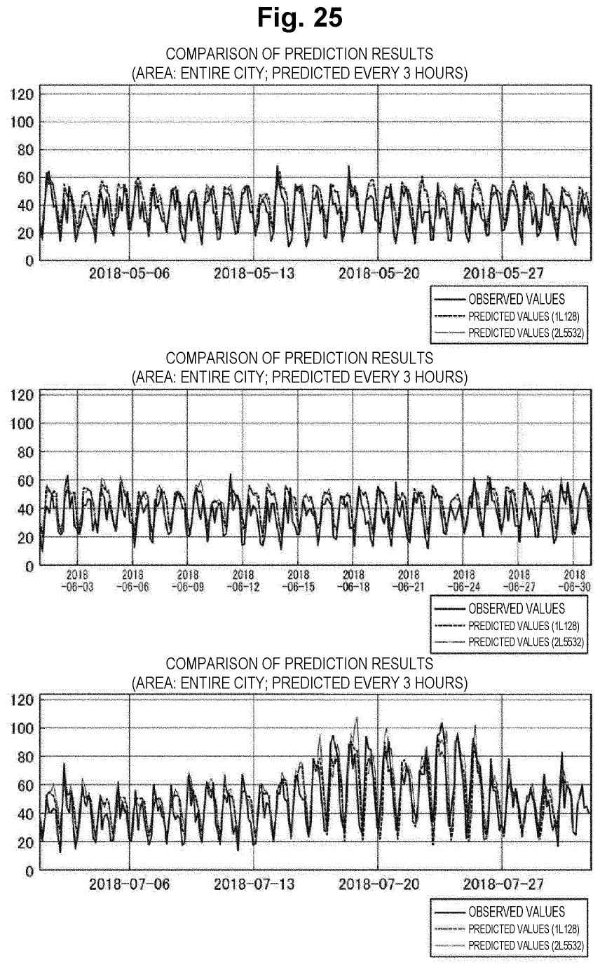

FIG. 26 is a flowchart showing the learning process performed by the emergency medical service demand prediction device 1 according to one embodiment of the present disclosure.

At step S601, in the emergency medical service demand prediction device 1, under the control of the transport data obtainment unit 21 and other functional units, the individual model learning units 541N receive the input of the various types of learning-purpose data. The various types of learning-purpose data include the learning-purpose data from the transport data pre-processing unit 22, the environment data from the environment data pre-processing unit 222, the region data from the region data pre-processing unit 322, and the feature values from the regional characteristic learning unit 421 expressing the regional characteristics.

At step S602, under the control of each of the individual model learning units 541N, the emergency medical service demand prediction device 1 learns each of the individual prediction models by using the various types of learning-purpose data. Among the individual prediction models, the prediction model at normal times performs the learning process by using the vector regression model, whereas the learning process of the prediction model for the outburst events is performed by using the deep learning model.

At step S603, under the control of the ensemble learning unit 542, the emergency medical service demand prediction device 1 performs an ensemble learning process by inputting the input data to the neural network, while varying the weights on the neural network in accordance with the seasons, the days of the week, the time spans of the day, and the meteorological situations at different times. Further, under the control of the ensemble learning unit 542, the emergency medical service demand prediction device 1 obtains and saves the integrated prediction model into the prediction model storage unit 32. The input data includes: the outputs of the individual prediction models included in the individual model learning units 541N; the emergency medical transport data including the training signal; and the meteorological condition and the date/time information serving as the indices of the weights used for determining the outputs. For the meteorological condition, the environment data corresponding to the position of the position information may be referenced. Alternatively, the learning process may be performed so that the weights are calculated on the basis of only the meteorological condition, without using the date/time information.

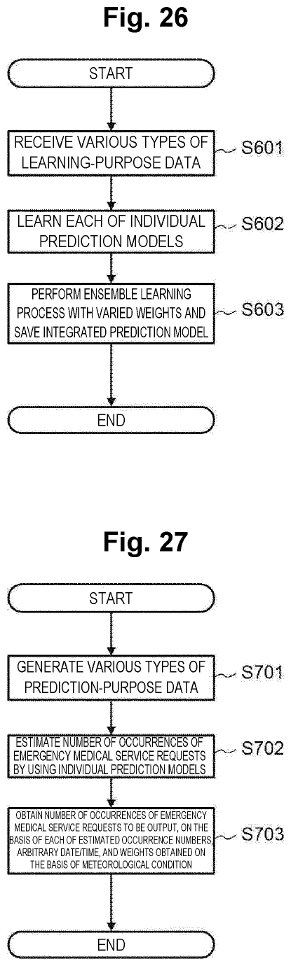

FIG. 27 is a flowchart showing a predicting process performed by the emergency medical service demand prediction device 1 according to one embodiment of the present disclosure. The predicting process is an example of the information processing method according to a technique of the present disclosure.

At step S701, under the control of the transport data pre-processing unit 22 and other functional units, the emergency medical service demand prediction device 1 generates various types of prediction-purpose data to be input to the prediction model. The various types of prediction-purpose data are generated by reading the trained categorization model stored in the categorization model storage unit 31 on the basis of the divided transport data and further calculating the number of occurrences per unit time period for each of the illness/injury groups. As a result, the illnesses and injuries are categorized into the illness/injury groups so that the frequency of occurrences of each of the illness/injury groups is calculated for each prescribed unit area and each prescribed unit time period. Further, the various types of prediction-purpose data include the environment data from the environment data pre-processing unit 222, the region data from the region data pre-processing unit 322, and the feature values from the regional characteristic learning unit 421 expressing the regional characteristics.

At step S702, under the control of the request occurrence number prediction unit 525, the emergency medical service demand prediction device 1 estimates the number of occurrences of the emergency medical service requests by using each of the individual prediction models, while using the various types of prediction-purpose data and the integrated prediction model stored in the prediction model storage unit 32. As for the prediction model at normal times, the number of occurrences of the emergency medical service requests is estimated by bringing an arbitrary date/time into association with the frequency of occurrences of each of the illness/injury groups calculated for each prescribed unit area and each prescribed unit time period, while using the prediction model at normal times. As for the prediction model for the outburst events, increases and decreases in the number of occurrences of the emergency medical service requests are estimated by bringing an arbitrary date/time into association with the frequency of occurrences of each of the illness/injury groups calculated for each prescribed unit area and each prescribed unit time period, while using the prediction model for the outburst events. As a result, for each of the situations, the frequency of occurrences of each of the illness/injury groups is calculated for each prescribed unit area and each prescribed unit time period.

At step S703, under the control of the request occurrence number prediction unit 525, the emergency medical service demand prediction device 1 obtains the number of occurrences of the emergency medical service requests to be output, by using the integrated prediction model and saves the obtained result into the prediction result storage unit 33. The number of occurrences of the emergency medical service requests to be output is obtained on the basis of the number of occurrences estimated by using the prediction model at normal times, the number of occurrences estimated by using the prediction model for the outburst events, the arbitrary date/time, and the weights obtained on the basis of the date/time information and the meteorological condition. As the date/time information and the meteorological condition, the information from the various types of prediction-purpose data may be used as appropriate. When the numbers of occurrences, the date/time information, and the meteorological condition are input to the neural network of the integrated prediction model, the weights on the neural network are calculated so that the neural network outputs the number of occurrences of the emergency medical service requests for each of the illness/injury groups corresponding to the weights. Alternatively, the weights may be calculated on the basis of only the meteorological condition, without using the date/time information.

### Advantageous Effects

As explained above, by using the emergency medical service demand prediction device 1 according to the present embodiment, it is possible to provide the technique for predicting the number of occurrences of the emergency medical service requests with a high level of precision and efficiently, while taking the conditions in the specific time period into account.

According the technique of the present disclosure, the optimal prediction models are trained by appropriately combining the meteorological condition and the date/time information, with respect to the time periods at normal times in the spring or the fall during which the increases and decreases are relatively mild and with respect to the time periods in the summer or the winter during which an increase in the number of emergency medical services, i.e., an increase in outburst events, is anticipated. By using the prediction models that are trained by appropriately combining the meteorological condition and the date/time information in this manner, it is possible to realize the prediction of the number of occurrences of the emergency medical transports with a high level of precision. Consequently, it is possible to obtain the predictions of the number of dispatches of the emergency medical staff, with a high level of precision throughout the year. In particular, it is possible to predict sudden increases in the summer and the winter from the first day onward. It is therefore expected to be possible to prevent delays in arrival at the emergency sites that may be caused by a depletion of emergency medical staff resources.

In addition, it is possible to carry out the present disclosure while modifying the types of the environment data and the region data, or the like, without departing from the scope of the present disclosure.

That is to say, the techniques of the present disclosure are not limited to the embodiments described above. At the stage of carrying out the present disclosure, it is possible to embody the techniques while modifying the constituent elements without departing from the scope thereof. Further, it is possible to structure various disclosed techniques by combining two or more of the constituent elements disclosed in the above embodiments, as appropriate. For example, some of the constituent elements described in any of the embodiments may be omitted. Also, it is also acceptable to combine, as appropriate, constituent elements from mutually-different embodiments.

### REFERENCE SIGNS LIST

1 Emergency medical service demand prediction device
10 Input/output interface unit
20 Control unit
21 Transport data obtainment unit
22 Transport data pre-processing unit
23 Illness/injury group learning unit
24 Prediction model learning unit
25 Request occurrence number prediction unit
26 Output control unit
30 Storage unit
31 Categorization model storage unit
32 Prediction model storage unit
33 Prediction result storage unit
221 Environment data obtainment unit
222 Environment data pre-processing unit
321 Region data obtainment unit
322 Region data pre-processing unit
421 Regional characteristic learning unit
434 Regional characteristic storage unit
524 Prediction model learning unit
525 Request occurrence number prediction unit
541N Individual model learning units
542 Ensemble learning unit.

1. A method for processing information, the method comprising:
obtaining at least patient attributes being attributes of patients, date/time information indicating dates and times of occurrences of emergency medical service requests, position information indicating places of the occurrences of the emergency medical service requests, and illness/injury information indicating illnesses and injuries that caused the emergency medical service requests;
categorizing the illnesses and injuries into illness/injury groups, on a basis of a likelihood of occurrences of the illnesses and injuries in the illness/injury information obtained by bringing the patient attributes, the position information, and the date/time information into association with one another;
estimating a quantity of occurrences of emergency medical service requests by bringing an arbitrary date/time into association with frequency of occurrences of each of the illness/injury groups calculated for each prescribed unit area and each prescribed unit time period, by using a first method; and
outputting the quantity of the occurrences of the emergency medical service requests.

2. The method according to claim 1, wherein increase and decrease in the quantity of the occurrences of the emergency medical service requests being different from time-series fluctuation are estimated by further bringing the arbitrary date/time into association with the frequency of occurrences of each of the illness/injury groups calculated for each prescribed unit area and each prescribed unit time period, by using a second method different from the first method,
the method further comprising:
obtaining the quantity of the occurrences of the emergency medical service requests to be output, on a basis of the quantity of the occurrences estimated by the first method, the increase and decrease in the quantity of the occurrences estimated by the second method, the arbitrary date/time, and a weight obtained on a basis of a meteorological condition corresponding at least to the position information.

3. The information processing method according to claim 2, wherein
the meteorological condition includes snowfall or rainfall of climate, as a condition, and wherein
the quantity of the occurrences of the emergency medical service requests to be output is obtained in such a manner that the more snowfall or rainfall there is in the climate, the larger is the weight applied to the quantity of the occurrences estimated by the second method.

4. A computer-readable non-transitory storage medium storing a computer-executable instructions that when executed by a processor cause the computer-executable instructions to execute a method comprising:
obtaining at least patient attributes being attributes of patients, date/time information indicating dates and times of occurrences of emergency medical service requests, position information indicating places of the occurrences of the emergency medical service requests, and illness/injury information indicating illnesses and injuries that caused the emergency medical service requests;
categorizing the illnesses and injuries into illness/injury groups, on a basis of a likelihood of occurrences of the illnesses and injuries in the illness/injury information obtained by bringing the patient attributes, the position information, and the date/time information into association with one another;
estimating a quantity of occurrences of emergency medical service requests by bringing an arbitrary date/time into association with frequency of occurrences of each of the illness/injury groups calculated for each prescribed unit area and each prescribed unit time period, by using a first method; and
outputting the quantity of the occurrences of the emergency medical service requests.

5. An information processing device comprising a processor configured to execute a method, a method comprising:
obtaining at least patient attributes being attributes of patients, date/time information indicating dates and times of occurrences of emergency medical service requests, position information indicating places of the occurrences of the emergency medical service requests, and illness/injury information indicating illnesses and injuries which caused the emergency medical service requests;
categorizing the illnesses and injuries into illness/injury groups, on a basis of a likelihood of occurrences of the illnesses and injuries in the illness/injury information obtained by bringing the patient attributes, the position information, and the date/time information into association with one another;
estimating a quantity of occurrences of emergency medical service requests by bringing an arbitrary date/time into association with frequency of occurrences of each of the illness/injury groups calculated for each prescribed unit area and each prescribed unit time period, by using a first method; and
outputting the quantity of the occurrences of the emergency medical service requests.

6. The method according to claim 1, wherein the quantity of occurrences of emergency medical service requests indicates an outburst of occurrences of emergency medical service requests.

7. The method according to claim 1, wherein the estimating uses a prediction model based on a neural network.

8. The method according to claim 1, wherein the categorizing uses a categorization model based on a neural network.

9. The computer-readable non-transitory storage medium according to claim 4, wherein increase and decrease in the quantity of the occurrences of the emergency medical service requests being different from time-series fluctuation are estimated by further bringing the arbitrary date/time into association with the frequency of occurrences of each of the illness/injury groups calculated for each prescribed unit area and each prescribed unit time period, by using a second method different from the first method,
the computer-executable instructions further execute a method comprising:
obtaining the quantity of the occurrences of the emergency medical service requests to be output, on a basis of the quantity of the occurrences estimated by the first method, the increase and decrease in the quantity of the occurrences estimated by the second method, the arbitrary date/time, and a weight obtained on a basis of a meteorological condition corresponding at least to the position information.

10. The computer-readable non-transitory storage medium according to claim 4, wherein the quantity of occurrences of emergency medical service requests indicates an outburst of occurrences of emergency medical service requests.

11. The computer-readable non-transitory storage medium according to claim 4, wherein the estimating uses a prediction model based on a neural network.

12. The computer-readable non-transitory storage medium according to claim 4, wherein the categorizing uses a categorization model based on a neural network.

13. The information processing device according to claim 5, wherein increase and decrease in the quantity of the occurrences of the emergency medical service requests being different from time-series fluctuation are estimated by further bringing the arbitrary date/time into association with the frequency of occurrences of each of the illness/injury groups calculated for each prescribed unit area and each prescribed unit time period, by using a second method different from the first method,
the processor further configured to execute a method comprising:
obtaining the quantity of the occurrences of the emergency medical service requests to be output, on a basis of the quantity of the occurrences estimated by the first method, the increase and decrease in the quantity of the occurrences estimated by the second method, the arbitrary date/time, and a weight obtained on a basis of a meteorological condition corresponding at least to the position information.

14. The information processing device according to claim 5, wherein the quantity of occurrences of emergency medical service requests indicates an outburst of occurrences of emergency medical service requests.

15. The information processing device according to claim 5, wherein the estimating uses a prediction model based on a neural network.

16. The information processing device according to claim 5, wherein the categorizing uses a categorization model based on a neural network.

17. The computer-readable non-transitory storage medium according to claim 9, wherein
the meteorological condition includes snowfall or rainfall of climate, as a condition, and wherein
the quantity of the occurrences of the emergency medical service requests to be output is obtained in such a manner that the more snowfall or rainfall there is in the climate, the larger is the weight applied to the quantity of the occurrences estimated by the second method.

18. The information processing device according to claim 13, wherein
the meteorological condition includes snowfall or rainfall of climate, as a condition, and wherein
the quantity of the occurrences of the emergency medical service requests to be output is obtained in such a manner that the more snowfall or rainfall there is in the climate, the larger is the weight applied to the quantity of the occurrences estimated by the second method.
